(19) United States
(12) Patent Application Publication
(10) Pub. No.: US 2005/0245540 A1
(43) Pub. Date: Nov. 3, 2005

(54) New methods

(75) Inventors: Nobuaki Takeshita, Osaka-shi (JP); Takayuki Inoue, Osaka-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka (JP)

(21) Appl. No.: 10/996,589

(22) Filed: Nov. 26, 2004

(30) Foreign Application Priority Data
Dec. 9, 2003 (AU) 2003906809

(51) Int. Cl. A61K 31/496; A61K 31/454

(52) U.S. Cl. 514/254.09; 514/323; 544/372; 546/200; 514/314; 546/167

(57) ABSTRACT

This invention relates to a method for preventing and/or treating bone diseases which comprises administering a potentiator of TGF-beta activity such as the compound of the formula [I] or pharmaceutically acceptable salts thereof to human being or animals. 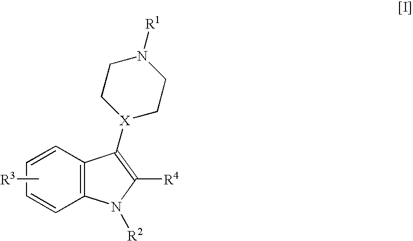 wherein
R¹ is hydrogen, etc.,
R² is optionally substituted ar(lower)alkyl or optionally substituted heterocyclic(lower)alkyl, etc.,
R³ is hydrogen, optionally substituted hydroxy, etc.,
R⁴ is hydrogen or lower alkyl, and X is CH or N.

### TECHNICAL FIELD

This invention relates to methods for preventing and/or treating bone diseases using a potentiator of Transforming Growth Factors beta (TGF-beta) activity.

### BACKGROUND ART

TGF-beta family including TGF-beta1, 2 and 3 as its subtypes consists of many structurally related peptides, which regulate a wide range of crucial cell growth and differentiation events, including early embryonic patterning and morphogenesis, sexual organ and bone/cartilage formation, wound healing and immunosuppression. TGF-beta is postulated to play a role in controlling bone density by regulating the fine balance between bone matrix deposition by osteoblasts and its resorption by osteoclasts. Evidence for the role of TGF-beta in regulating bone mineral density comes from a number of observations, for example the growth factors including TGF-beta promote osteoblast proliferation or differentiation (Clin Orthop. 30, 263, (1991)) and injections of TGF-beta1 decrease osteoclastic resorption in rats (J. Bone Miner Res 971, 10, (1995)). TGF-beta from bone or platelets stimulates bone cell replication in vitro (Endocrinology 2306, 119, (1986)). TGF-beta stimulates local periosteal woven bone formation in vivo (Endocrinology 2991, 124, (1989)). The rate of bone formation is altered in TGF-beta1 knockout mice (Bone 87, 23, (1998)). Administration of TGF-beta corrects the bone density deficiency in elderly mice with osteoporosis (J. Cellular Biochemistry 379, 73, (1999)) and the TGF-beta family is expressed over a 28-day period of fracture healing in mouse (J. Bone Miner Res 513, 17, (2002)).

WO98/53821 discloses 1,2,3,6-tetrahydropyridine derivatives which are useful for the treatment of bone diseases, for examples osteoporosis and bone fractures, by increasing the level of TGF-beta1. JP06-239815 discloses 2-aminoethoxybenzene derivatives which are useful for the treatment of osteoporosis as a TGF-beta production promoter. WO03/000257 discloses oxazole or thiazole compounds which are TGF-beta superfamily production/secretion promoter and useful as a preventive or remedy for autonomic neuropathy, bladder function disorders, auditory disorder and bone diseases.

On the other hand, some indole compounds are known, for example, in WO92/00070, WO92/13856, WO94/24127, WO99/17773, WO99/55694, WO99/58525, WO00/75130, WO00/78716, EP0200322, EP708102, EP714894 and EP722942. However, it is not known that these indole compounds are useful for preventing and/or treating bone diseases.

### DISCLOSURE OF INVENTION

This invention relates to a method for preventing and/or treating bone diseases which comprises administering a potentiator of TGF-beta activity such as indole compounds or pharmaceutically acceptable salts thereof to human being or animals.

Accordingly, one object of this invention is to provide a method for preventing and/or treating bone diseases which comprises administering a potentiator of TGF-beta activity such as indole compounds mentioned below or pharmaceutically acceptable salts thereof to human being or animals.

Another object of this invention is to provide use of a potentiator of TGF-beta activity for manufacturing a medicament for treating and/or preventing bone diseases.

A further object of this invention is to provide an agent for preventing and/or treating bone diseases, which comprises a potentiator of TGF-beta activity.

A still further object of this invention is to provide new indole compounds or pharmaceutically acceptable salts thereof which are useful for preventing and/or treating bone diseases as a potentiator of TGF-beta activity.

A still further object of this invention is to provide a new pharmaceutical composition containing, as an active ingredient, said new indole compounds or pharmaceutically acceptable salts thereof.

This invention is directed to methods for preventing and/or treating bone diseases which comprises administering a potentiator of TGF-beta activity such as an indole compounds of the formula [I]: 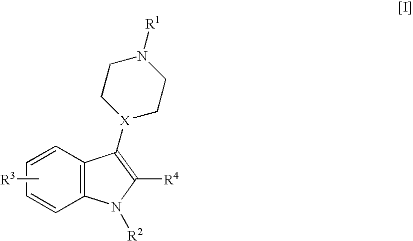
wherein
R¹ is hydrogen, acyl, lower alkyl, optionally substituted ar(lower)alkyl or a group of the formula:
   -A-B,
in which A is alkylene having one to ten carbon atoms, and
   B is amino optionally substituted with acyl or lower alkyl,
R² is hydrogen, lower alkyl, cyclo(lower)alkyl(lower)alkyl, acyl, optionally substituted ar(lower)alkyl or optionally substituted heterocyclic-(lower)alkyl,
R³ is hydrogen, optionally substituted hydroxy, optionally substituted amino or cyano,
R⁴ is hydrogen or lower alkyl, and
X is CH or N,
   or its pharmaceutically acceptable salt to human being or animals.

A preferred embodiments of the compound [I] used in this invention are as follows:
R¹ is
   (1) hydrogen,
   (2) acyl,
   (3) lower alkyl,
   (4) ar(lower)alkyl optionally substituted with lower alkoxy, or
   (5) a group of the formula:
      -A-B
   wherein A is alkylene having one to ten carbon atoms, and
      B is amino optionally substituted with acyl or lower alkyl,
R² is
   (1) hydrogen,
   (2) lower alkyl,
   (3) cyclo(lower)alkyl(lower)alkyl,
   (4) acyl,
   (5) ar(lower)alkyl optionally substituted with one or more substituents selected from the group consisting of
      lower alkyl,
      halo(lower)alkyl,
      nitro,
      amino,
      halogen,
      cyano,
      hydroxy,
      lower alkoxy,
      lower alkylthio,
      aryl optionally substituted with lower alkyl or
      halo(lower)alkyl,
      acyl,
      ar(lower)alkyl,
      lower alkylenedioxy,
      aryloxy,
      ar(lower)alkenyl, and
      ar(lower)alkoxy, or
   (6) heterocyclic(lower)alkyl optionally substituted with one or more substituents selected from the group consisting of
      lower alkyl,
      halo(lower)alkyl,
      nitro,
      amino,
      halogen,
      cyano,
      hydroxy,
      lower alkoxy,
      lower alkylthio,
      aryl optionally substituted with lower alkyl or
      halo(lower)alkyl,
      acyl,
      ar(lower)alkyl,
      lower alkylenedioxy,
      aryloxy,
      ar(lower)alkenyl, and
      ar(lower)alkoxy,
R³ is
   (1) hydrogen,
   (2) hydroxy,
   (3) aroyloxy or ar(lower)alkoxy, each of which is optionally substituted with one or more substituents selected from the group consisting of
      halogen,
      acyl,
      aryl,
      lower alkyl, and
      halo(lower)alkyl,
   (4) lower alkoxy,
   (5) cyclo(lower)alkyl(lower)alkoxy,
   (6) amino optionally substituted with lower alkyl or acyl, or
   (7) cyano,
R⁴ is hydrogen or lower alkyl, and
X is CH or N,
   or its pharmaceutically acceptable salt.

Other preferred embodiments of the compound [I] used in this invention are as follows:
R¹ is
   (1) hydrogen,
   (2) lower alkanoyl,
   (3) lower alkoxycarbonyl,
   (4) amino(lower)alkanoyl,
   (5) lower alkoxycarbonylamino(lower)alkanoyl,
   (6) lower alkylsulfonyl,
   (7) lower alkyl,
   (8) phenyl(lower)alkyl optionally substituted with lower alkoxy, or
   (9) a group of the formula:
      -A-B
   wherein A is alkylene having one to ten carbon atoms, and B is amino optionally substituted with one or two substituents selected from the group consisting of
      lower alkyl,
      lower alkanoyl,
      lower alkoxycarbonyl,
      benzoyl, and
      phthaloyl,
R² is
   (1) hydrogen,
   (2) lower alkyl,
   (3) cyclo(lower)alkyl(lower)alkyl,
   (4) benzoyl optionally substituted with lower alkyl or halo(lower)alkyl,
   (5) lower alkylsulfonyl,
   (6) phenylsulfonyl,
   (7) phenyl(lower)alkyl, naphthyl(lower)alkyl or anthryl-(lower)alkyl, each of which is optionally substituted with one or more substituents selected from the group consisting of
      lower alkyl,
      halo(lower)alkyl,
      nitro,
      amino,
      halogen,
      hydroxy,
      cyano,
      lower alkoxy,
      lower alkylthio,
      phenyl optionally substituted with lower alkyl or
      halo(lower)alkyl,
      lower alkoxycarbonyl,
      lower alkylsulfonyl,
      carboxy,
      N-phenylcarbamoyl,
      N-phenyl-N-(lower)alkylcarbamoyl,
      phenyl(lower)alkyl,
      lower alkylenedioxy,
      phenoxy,
      phenyl(lower)alkenyl, and
      phenyl(lower)alkoxy,
   (8) quinolyl(lower)alkyl or oxadiazolyl(lower)alkyl, each of which is optionally substituted with one or more substituents selected from the group consisting of
      lower alkyl,
      halo(lower)alkyl,
      nitro,
      amino,
      halogen,
      hydroxy,
      cyano,
      lower alkoxy,
      lower alkylthio,
      phenyl optionally substituted with lower alkyl or
      halo(lower)alkyl,
      lower alkoxycarbonyl,
      lower alkylsulfonyl,
      carboxy,
      N-phenylcarbamoyl,
      N-phenyl-N-(lower)alkylcarbamoyl,
      phenyl(lower)alkyl,
      lower alkylenedioxy,
      phenoxy,
      phenyl(lower)alkenyl, and
      phenyl(lower)alkoxy,
R³ is
   (1) hydrogen,
   (2) hydroxy,
   (3) benzoyloxy,
   (4) phenyl(lower)alkoxy or naththyl(lower)alkoxy, each of which is optionally substituted with one or more substituents selected from the group consisting of
      halogen,
      phenyl,
      lower alkyl,
      halo(lower)alkyl, and
      lower alkoxycarbonyl,
   (5) lower alkoxy,
   (6) cyclo(lower)alkyl(lower)alkoxy,
   (7) amino optionally substituted with lower alkyl or benzoyl, or
   (8) cyano,
R⁴ is hydrogen or lower alkyl and
X is CH or N,
or its pharmaceutically acceptable salt.
   Other preferred embodiments of the compound [I ] used in this invention are as follows:
R¹ is hydrogen or lower alkoxycarbonyl,
R² is phenyl(lower)alkyl optionally substituted with one or more substituents selected from the group consisting of
   lower alkyl,
   halo(lower)alkyl,
   nitro,
   amino,
   halogen,
   hydroxy,
   cyano,
   lower alkoxy,
   lower alkylthio,
   phenyl,
   lower alkoxycarbonyl,
   lower alkylsulfonyl,
   carboxy,
   N-phenylcarbamoyl,
   N-phenyl-N-(lower)alkylcarbamoyl,
   phenyl(lower)alkyl,
   lower alkylenedioxy,
   phenoxy,
   phenyl(lower)alkenyl, and
   phenyl(lower)alkoxy,
R³ is
   (1) hydrogen,
   (2) hydroxy,
   (3) benzoyloxy,
   (4) phenyl(lower)alkoxy optionally substituted with one or more substituents selected from the group consisting of
      halogen,
      phenyl,
      lower alkyl,
      halo(lower)alkyl, and
      lower alkoxycarbonyl,
   (5) lower alkoxy,
   (6) cyclo(lower)alkyl(lower)alkoxy,
   (7) amino optionally substituted with lower alkyl or benzoyl, or
   (8) cyano,
R⁴ is hydrogen, and
X is CH,
   or its pharmaceutically acceptable salt.
   Other preferred embodiments of the compound [I] used in this invention are as follows:
R¹ is a group of the formula:
   -A-B
wherein A is alkylene having one to ten carbon atoms, and B is amino optionally substituted with one or two substituents selected from the group consisting of
   lower alkyl,
   lower alkanoyl,
   lower alkoxycarbonyl,
   benzoyl, and
   phthaloyl,
R² is
   (1) hydrogen,
   (2) lower alkyl,
   (3) cyclo(lower)alkyl(lower)alkyl,
   (4) benzoyl optionally substituted with lower alkyl or halo(lower)alkyl,
   (5) lower alkylsulfonyl,
   (6) phenylsulfonyl,
   (7) phenyl(lower)alkyl optionally substituted with one or more substituents selected from the group consisting of
      lower alkyl,
      halo(lower)alkyl,
      nitro,
      amino,
      halogen,
      hydroxy,
      cyano,
      lower alkoxy,
      lower alkylthio,
      phenyl,
      lower alkoxycarbonyl,
      lower alkylsulfonyl,
      carboxy,
      N-phenylcarbamoyl,
      N-phenyl-N-(lower)alkylcarbamoyl,
      phenyl(lower)alkyl,
      lower alkylenedioxy,
      phenoxy,
      phenyl(lower)alkenyl, and
      phenyl(lower)alkoxy,
R³ is
   (1) hydrogen,
   (2) hydroxy,
   (3) benzoyloxy,
   (4) phenyl(lower)alkoxy optionally substituted with one or more substituents selected from the group consisting of
      halogen,
      phenyl,
      lower alkyl,
      halo(lower)alkyl, and
      lower alkoxycarbonyl,
   (5) lower alkoxy,
   (6) cyclo(lower)alkyl(lower)alkoxy,
   (7) amino optionally substituted with lower alkyl or benzoyl, or
   (8) cyano,
R⁴ is hydrogen, and
X is CH,
   or its pharmaceutically acceptable salt.

In the above and subsequent description of the present specification and claims, suitable examples and illustrations of the various definitions to be included within the scope of the invention are explained in detail as follows.

Suitable “aryl” and aryl moiety such as in the terms “ar(lower)alkyl”, “aryloxy”, etc., may include phenyl, naphthyl, anthryl, or the like, in which preferable one is phenyl.

Suitable “aroyloxy” may include benzoyloxy or naphthoyloxy, or the like.

Suitable “heterocyclic group” and heterocyclic moiety such as in the term “heterocyclic(lower)alkyl”, etc., may include saturated or unsaturated, monocyclic or polycyclic heterocyclic group containing at least one nitrogen atom. And especially preferable heterocyclic ring containing nitrogen may be ones such as unsaturated 3 to 8-membered heteromonocyclic group containing 1 to 4 nitrogen atom(s), for example, pyrrolyl, pyrrolinyl, imidazolyl, pyrazolyl, pyridyl and its N-oxide, pyrimidinyl, pyrazinyl, dihydropyridazinyl, tetrahydropyridazinyl, triazolyl (e.g., 1H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), dihydrotriazinyl (e.g., 4,5-dihydro-1,2,4-triazinyl, 2,5-dihydro-1,2,4-triazinyl, etc.), etc.,; saturated 3 to 8-membered heteromonocyclic group containing 1 to 4 nitrogen atom(s), for example, pyrrolidinyl, imidazolidinyl, piperidinyl, piperazinyl, azacycloheptyl, azacyclooctyl, perhydroazepinyl, etc.,; unsaturated condensed heterocyclic group containing 1 to 4 nitrogen atom(s), for example, indolyl, 2,3-dihydroindolyl, isoindolyl, indolinyl, indazolyl, isoindolinyl, indolizinyl, benzimidazolyl, quinolyl, 1,2,3,4-tetrahydroquinolyl, isoquinolyl, indazolyl, benzotriazolyl, tetrazolopyridyl, tetrazolopyridazinyl (e.g., tetrazolo[1,5-b]pyridazinyl,etc.,), dihydrotriazolopyridazinyl, etc.,: unsaturated 3 to 8-membered heteromonocyclic group containing 1 to 2 oxygen atom(s) and 1 to 3 nitrogen atom(s), for example, oxazolyl, isoxazolyl, dihydroisoxazolyl, oxadiazolyl (e.g., 1,2,4-oxadiazolyl, 1,3,4-oxadiazolyl, 1,2,5-oxadiazolyl, etc.,), etc.,; saturated 3 to 8-membered heteromonocyclic group containing 1 to 2 oxygen atom(s) and 1 to 3 nitrogen atom(s), for example, morpholino, etc.,; unsaturated condensed heterocyclic group containing 1 to 2 oxygen atom(s) and 1 to 3 nitrogen atom(s), for example, benzoxazolyl, benzoxadiazolyl, etc.,; unsaturated 3 to 8-membered heteromonocyclic group containing 1 to 2 sulfur atom(s), for example, thienyl, thiepinyl, etc.,; unsaturated 3 to 8-membered heteromonocyclic group containing 1 to 2 sulfur atom(s) and 1 to 3 nitrogen atom(s), for example, thiazolyl, isothiazolyl, thiazolinyl, thiadiazolyl (e.g., 1,2,4-thiadiazolyl, 1,3,4-thiadiazolyl, 1,2,5-thiadiazolyl, 1,2,3-thiadiazolyl), etc.,; saturated 3 to 8-membered heteromonocyclic group containing 1 to 2 sulfur atom(s) and 1 to 3 nitrogen atom(s), for example, thiazolidinyl, etc.,; unsaturated condensed hetero mono cyclic group containing 1 to 2 sulfur atom(s) and 1 to 3 nitrogen atom(s), for example, benzothiazolyl, benzothiadiazolyl, etc., or the like.

Suitable “halogen” may include fluorine, chlorine, bromine, or iodine.

The term “lower” is intended to mean 1 to 6 carbon atom(s), unless otherwise indicated.

In this respect, the term “lower” in lower alkenyl moiety in the various definitions is intended to mean 2 to 6 carbon atoms.

Further, the term “lower” in cyclo(lower)alkyl moiety and cyclo(lower)alkoxy moiety in the various definitions is intended to mean 3 to 6 carbon atoms.

Suitable “lower alky” and lower alkyl moieties such as in the terms “lower alkylthio”, “ar(lower)alkyl”, etc., may include straight or branched one having 1 to 6 carbon atom(s), such as methyl, ethyl, propyl, isopropyl, butyl, isobutyl, sec-butyl, t-butyl, pentyl, t-pentyl, hexyl or the like, preferably one having 1 to 4 carbon atoms.

Suitable “halo(lower)alkyl” may include monofluoromethyl, difluoromethyl, trifluoromethyl, 1,2-dichloroethyl, or the like.

Suitable “cyclo(lower)alkyl” and cyclo(lower)alkyl moiety such as in the term “cyclo(lower)alkyl(lower)alkyl”, etc., may include cyclopropyl, cyclopentyl, cyclobutyl, cyclohexyl, or the like.

Suitable “lower alkenyl” and lower alkenyl moiety such as in the term “ar(lower)alkenyl”, etc., may include straight or branched one having 2 to 6 carbon atoms, such as ethenyl, propenyl, butenyl, pentenyl, hexenyl, or the like.

Suitable “lower alkoxy” and lower alkoxy moiety such as in the term “ar(lower)alkoxy”, etc., may include methoxy, ethoxy propoxy, isopropoxy, butoxy, isobutoxy, t-butoxy, pentyloxy, t-pentyloxy, hexyloxy, or the like.

Suitable “lower alkylenedioxy” may include such as methylenedioxy, ethylenedioxy, propylenedioxy, and the like.

Suitable “alkylene” may include straight or branched having one to ten carbon atom(s), such as methylene, ethylene, trimethylene, tetramethylene, pentamethylene, hexamethylene, heptamethylene, octamethylene, nonamethylene, decamethylene, and the like, in which preferable one is straight or branched having six to ten carbon atoms such as hexamethylene, heptamethylene, octamethylene, nonamethylene, and decamethylene.

Suitable “acyl” and acyl moiety such as in the term of “acylamino”, etc., may include aliphatic acyl group and acyl group containing an aromatic or heterocyclic ring.

And, suitable examples of the said acyl may be lower alkanoyl (e.g. formyl, acetyl, propionyl, butyryl, isobutyryl, valeryl, isovaleryl, oxalyl, succinyl, pivaloyl, etc.); amino(lower)alkanoyl, lower alkyloxycarbonylamino(lower)-alkanoyl, lower alkenoyl (e.g., propenoyl, 2-methylpropenoyl or butenoyl, or the like,); lower alkoxycarbonyl (e.g., methoxycarbonyl, ethoxycarbonyl propoxycarbonyl, isopropoxycarbonyl, butoxycarbonyl, isobutoxycarbonyl, t-butoxycarbonyl. pentyloxycarbonyl, t-pentyloxycarbonyl or hexyloxycarbonyl, or the like, preferably t-butoxycarbonyl); aroyl (e.g., benzoyl, naphthoyl, phthaloyl, etc.); heterocycliccarbonyl (e.g., pyridylcarbonyl, morpholinocarbonyl, etc.); cyclo(lower)- alkanecarbonyl (cyclopropanecarbonyl, cyclobutanecarbonyl, cyclohexanecarbonyl, etc.), carboxy, carbamoyl which may be substituted with lower alkyl or aryl (e.g. N-phenylcarbamoyl, N-phenyl-N-(lower)alkylcarbamoyl, etc.), lower alkylsulfonyl (e.g., methanesulfonyl, etc.), arylsulfonyl (e.g., phenylsulfonyl, etc.), or the like.

Suitable “amino protective group” may include acyl such as lower alkanoyl (e.g. formyl, acetyl, propionyl, pivaloyl, hexanoyl, etc.), mono- (or di- or tri-) halo (lower) alkanoyl group (e.g. chloroacetyl, bromoacetyl, dichloroacetyl, trifluoroacetyl, etc.), lower alkoxycarbonyl group, (e.g. methoxycarbonyl, ethoxycabonyl, propoxycarbonyl, tert-butoxycarbonyl, etc.), carbamoyl group, aroyl group (e.g. benzoyl, toluoyl, naphthoyl, etc.), ar (lower) alkanoyl group (e.g. phenylacetyl. phenylpropionyl, etc.), aryloxycarbonyl group (e.g. phenoxycarbonyl, naphthyloxycarbonyl, etc.), aryloxy (lower) alkanoyl group (e.g. phenoxyacetyl, phenoxypropionyl, etc.), arylglyoxyloyl group, (e.g. phenylglyoxyloyl, naphthylglyoxyloyl, etc.) and ar (lower) alkoxycarbonyl group which may have suitable substituent(s), (e.g. benzyloxycarbonyl phenethyloxycarbonyl, p-nitrobenzyloxycarbonyl, etc.) or ar(lower) alkyl group such as mono- (or di- or tri-) phenyl (lower) alkyl (e.g. benzyl, phenethyl, benzhydryl, trityl, etc.), or the like.

Suitable “substituents” in the terms “optionally substituted ar(lower)alkyl”, “optionally substituted heterocyclic(lower)alkyl”, etc., may be lower alkyl, halo(lower)alkyl, nitro, amino, halogen, cyano, hydroxy, lower alkoxy, lower alkylthio, aryl, halo(lower)alkyl, acyl, ar(lower)alkyl, lower alkylenedioxy, aryloxy, ar(lower)alkenyl, ar(lower)alkoxy or the like.

Suitable “substituents” in the term “optionally substituted hydroxy” may be aroyloxy, ar(lower)alkoxy, lower alkoxy, cyclo(lower)alkyl(lower)alkoxy, or the like.

Suitable “substituents” in the term “optionally substituted amino” may be lower alkyl, acyl, or the like.

Suitable “leaving group” may include halogen as exemplified above, acyloxy (e.g. acetyloxy, methanesulfonyloxy, p-toluenesulfonyloxy), and the like.

“A potentiator of Transforming Growth Factors beta (TGF-beta) activity” is defined as a substance which potentiates activity of TGF-beta in combination with TGF-beta. It is included that the potentiator administrated alone potentiates endogenous TGF-beta activity in human being or animals.

Suitable salts of the compounds [I] are pharmaceutical acceptable conventional non-toxic salts and include a metal salt such as an alkali metal salt (e.g., sodium salt, potassium salt, etc.), an ammonium salt, an organic base salt (e.g., trimethylamine, salt, triethylamine salt, pyridine salt, picoline salt, dicyclohexylamine salt, etc.), an organic acid salt (e.g., acetate, maleate, tartrate, methanesulfonate, benzenesulfonate, formate, toluenesulfonate, trifluoroacetate, etc.), on inorganic acid salt (e.g., hydrochloride, hydrobromide, sulfate, phosphate, etc.) or a salt with an amino acid (e.g., arginine, aspartic acid, glutamic acid, etc.), or the like.

The compounds of formula [I] may include one or more stereoisomers and geometrical isomers due to asymmetric carbon atoms and double bonds, and all of such isomers and mixture thereof are included within the scope of this invention.

The compounds of formula [I] may also exist in tautomeric forms and the invention includes both mixtures and separate individual tautomers.

The compounds of formula [I] and a salt thereof can be in a form of a solvate, which is included within the scope of the present invention. The solvate preferably includes a hydrate and an ethanolate.

Also included in the scope of the present invention are radiolabelled derivatives of compounds of formula [I] which are suitable for biological studies, and any form of the crystal of the compounds of formula [I].

According to the present invention, the compound [I] or a pharmaceutically acceptable salt thereof can be, for example, prepared by the following processes. 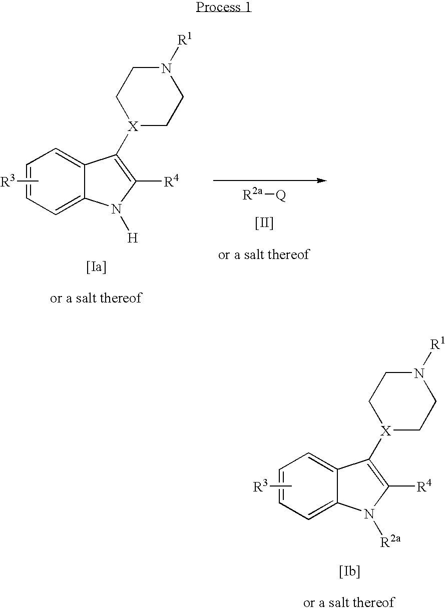
wherein
R^{2a} is lower alkyl, cyclo(lower)alkyl(lower)alkyl, optionally substituted ar(lower)alkyl or optionally substituted heterocyclic(lower)alkyl,
Q is a leaving group, and
R¹, R³, R⁴ and X are each as defined above.

The compound [Ib] or a salt thereof can be prepared by reacting a compound [Ia] or a salt thereof with a compound [II] or a salt thereof.

Suitable salts of the compound [II] may be the same as those exemplified for the compound [I].

The reaction may be carried out in the presence of organic or inorganic base.

Suitable organic bases include tri(lower)alkylamine [e.g., triethylamine or N,N-diisopropylethylamine], alkyl lithium [e.g., methyl lithium or butyl lithium], lithium diisopropylamide, lithium hexamethyldisilazido, pyridine, N-(lower)alkylmorphorine [e.g., N-methylmorphorine] and the like.

Suitable inorganic bases include an alkali metal [e.g., sodium or potassium], an alkali metal hydroxide [e.g., sodium hydroxide or potassium hydroxide], an alkali metal hydrogen carbonate [e.g., sodium hydrogen carbonate or potassium hydrogen carbonate], an alkali metal carbonate [e.g., sodium carbonate], an alkali metal hydride [e.g., sodium hydride or potassium hydride] and the like.

The reaction is usually carried out in a conventional solvent such as water, acetone, alcohol [e.g., methanol, ethanol, isopropyl alcohol, etc.], tetrahydrofuran, dioxane, toluene, methylene chloride, chloroform, N,N-dimethylformamide, or any other organic solvents which do not adversely affect the reaction, or the mixture thereof.

The reaction temperature is not critical and the reaction is usually carried out under cooling to warming. 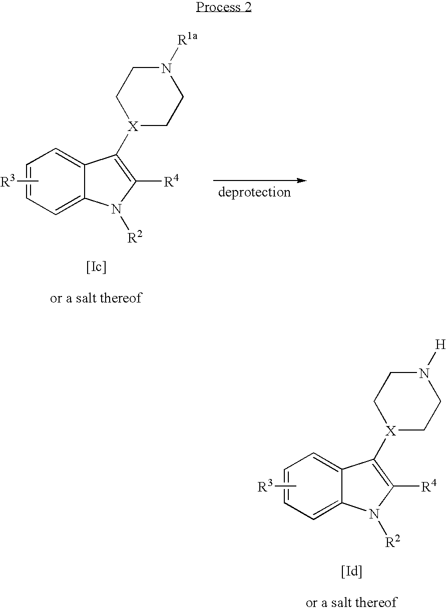
wherein
R^{1a} is an amino protective group, and
R², R³, R⁴ and X are each as defined above.

The compound [Id] or a salt thereof can be prepared by subjecting a compound [Ic] or a salt thereof to deprotection reaction.

The deprotection reaction is carried out by the routine procedure for removing an amino protecting group, for example by hydrolysis or reduction.

The reaction is carried out by hydrolysis which is preferably carried out in the presence of a base or an acid (inclusive of a Lewis acid).

The preferred base includes inorganic and organic bases such as alkali metals (e.g. sodium, potassium, etc.), alkaline earth metals (e.g. magnesium, calcium, etc.), the hydroxides, carbonates or hydrogen-carbonates of said metals, alkali metal alkoxides (e.g. sodium methoxide, sodium ethoxide, potassium t-butoxide, etc.), tri(lower)alkylamines (e.g. trimethylamine, triethylamine, etc.), pyridine, or the like.

The preferred acid includes organic acids (e.g. formic acid, acetic acid, propionic acid, trichloroacetic acid, trifluoroacetic acid, etc.) and inorganic acids (e.g. hydrochloric acid, hydrobromic acid, sulfuric acid, etc.).

This hydrolysis reaction is generally conducted in the common solvent such as water, alcohol (e.g. methanol, ethanol, etc.), diethyl ether, dioxane, tetrahydrofuran, dichloromethane, ethyl acetate, etc., a mixture of such solvents, or a suitable other organic solvent that does not interfere with the reaction. When the above-mentioned base or acid is a liquid, the base or acid may be used as the solvent as well.

There is no particular limitation on the reaction temperature but the reaction is generally conducted under cooling, at room temperature, or at elevated temperature.

The reduction method which can be applied to the deprotection reaction includes catalytic reduction.

The preferred catalyst which can be used for the catalytic reduction includes but is not limited to the common catalysts such as platinum catalysts (e.g. platinum oxide, etc.), palladium catalysts (e.g. palladium oxide, palladium-carbon, etc.).

The reduction reaction is generally carried out in a solvent such as water, alcohol (e.g. methanol, ethanol, propanol, etc.), N,N-dimethylformamide, diethyl ether, dioxane, tetrahydrofuran, etc., a mixture thereof or any other organic solvents which do not adversely affect the reaction.

The temperature of the reaction is not critical and the reaction is usually carried out from under cooling to heating under the pressure of 1-5 atmosphere. 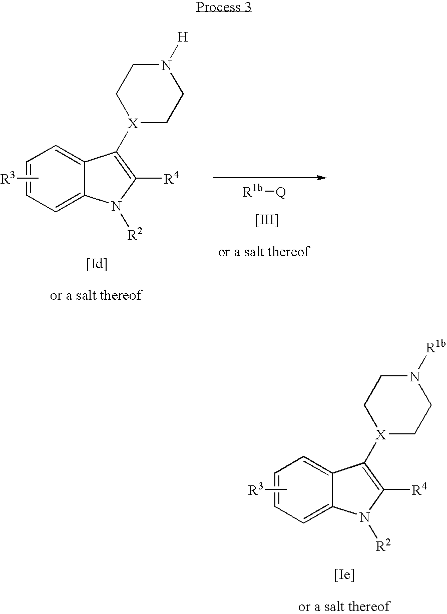
wherein
R^{1b} is lower alkyl, optionally substituted ar(lower)alkyl or a group of the formula:
   -A-B,
in which A and B are each as defined above, and
R², R³, R⁴, X and Q are each as defined above.

The compound [Ie] or a salt thereof can be prepared by reacting a compound [Id] or a salt thereof with a compound [III] or a salt thereof.

The suitable salts of the compound [III] may be the same as those exemplified for the compound [I].

The reaction can be carried out in substantially the same manner as Process 1, and therefore the reaction mode and reaction condition of this reaction are to be referred to those explained in Process 1.

Another aspect of this invention is directed to a use of a potentiator of TGF-beta activity such as the compound [I] or its pharmaceutically acceptable salt for manufacturing medicament for treating and/or preventing bone diseases.

Other aspect of this invention is directed to an agent for preventing and/or treating bone diseases, which comprises a potentiator of TGF-beta activity such as the compound [I] or its pharmaceutically acceptable salt.

Other aspect of this invention is directed to a novel compound of the formula [If]: 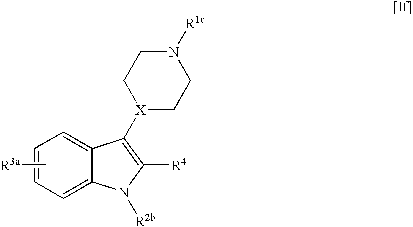
wherein
R^{1c} is hydrogen or acyl,
R^{2b} is optionally substituted ar(lower)alkyl,
R^{3a} is hydrogen, hydroxy, lower alkoxy, cyano, amino or acylamino,
R⁴ is hydrogen or lower alkyl, and
X is CH or N,
   or its pharmaceutically acceptable salt.

Preferred embodiments of the compound [If] are as follows:
R^{1c} is hydrogen or lower alkoxycarbonyl,
R^{2b} is phenyl(lower)alkyl optionally substituted with one or more substituents selected from the group consisting of
   lower alkyl,
   halo(lower)alkyl,
   nitro,
   amino,
   halogen,
   hydroxy,
   cyano,
   lower alkoxy,
   lower alkylthio,
   phenyl,
   lower alkoxycarbonyl,
   lower alkylsulfonyl,
   carboxy,
   N-phenylcarbamoyl,
   N-phenyl-N-(lower)alkylcarbamoyl,
   phenyl(lower)alkyl,
   lower alkylenedioxy,
   phenoxy,
   phenyl(lower)alkenyl, and
   phenyl(lower)alkoxy,
R^{3a} is
   (1) hydrogen,
   (2) hydroxy,
   (3) lower alkoxy,
   (4) amino optionally substituted with benzoyl, or
   (5) cyano,
R⁴ is hydrogen, and
X is CH,
   or its pharmaceutically acceptable salt.

More preferred embodiments of the compound [If] are as follows
R^{1c} is hydrogen or lower alkoxycarbonyl,
R^{2b} is phenyl(lower)alkyl optionally substituted with one or more substituents selected from the group consisting of
   lower alkyl,
   lower alkoxy,
   lower alkylthio,
   lower alkylsulfonyl,
   halogen,
   lower alkoxycarbonyl,
   nitro,
   halo(lower)alkyl, and
   lower alkylenedioxy,
R^{3a} is hydrogen or cyano,
R⁴ is hydrogen, and
X is CH,
   or its pharmaceutically acceptable salt.

Other aspect of this invention is directed to a novel compound of the formula [Ig]: 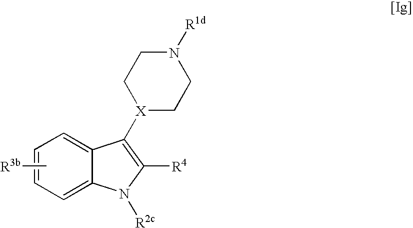
wherein
R^{1d} is a group of the formula
   -A¹-B¹
in which
   A¹ is alkylene having six to ten carbon atoms, and
   B¹ is amino optionally substituted with acyl or lower alkyl,
R^{2c} is hydrogen or optionally substituted ar(lower)alkyl,
R^{3b} is hydrogen, hydroxy or lower alkoxy,
R⁴ is hydrogen or lower alkyl, and
X is CH or N,
   or its pharmaceutically acceptable salt.

Preferred embodiments of the compound [Ig] are as follows:
R^{1d} is a group of the formula:
   -A¹-B¹
in which
   A¹ is alkylene having seven to ten carbon atoms, and
   B¹ is amino optionally substituted with one or two substituents selected from the group consisting of
      lower alkyl,
      lower alkanoyl,
      lower alkoxycarbonyl,
      benzoyl, and
      phthaloyl,
R^{2c} is
   (1) hydrogen,
   (2) phenyl(lower)alkyl optionally substituted with one or more substituents selected from the group consisting of
      lower alkyl,
      halo(lower)alkyl,
      nitro,
      amino,
      halogen,
      hydroxy,
      cyano,
      lower alkoxy,
      lower alkylthio,
      phenyl,
      lower alkoxycarbonyl,
      lower alkylsulfonyl,
      carboxy,
      N-phenylcarbamoyl,
      N-phenyl-N-(lower)alkylcarbamoyl,
      phenyl(lower)alkyl,
      lower alkylenedioxy,
      phenoxy,
      phenyl(lower)alkenyl, and
      phenyl(lower)alkoxy,
R^{3b} is
   (1) hydrogen,
   (2) hydroxy, or
   (3) lower alkoxy,
R⁴ is hydrogen, and
X is CH,
   or its pharmaceutically acceptable salt.

More preferred embodiments of the compound [Ig] are as follows:
R^{1d} is a group of the formula:
   -A¹-B¹
in which
   A¹ is straight alkylene having seven to ten carbon atoms, and
   B¹ is lower alkanoylamino or lower alkoxycarbonylamino,
R^{2c} is hydrogen,
R^{3b} is hydrogen, hydroxy or lower alkoxy,
R⁴ is hydrogen, and
X is CH,
   or its pharmaceutically acceptable salt .

The compounds [If] and [Ig] or salt thereof can be, for example, prepared by the following processes. 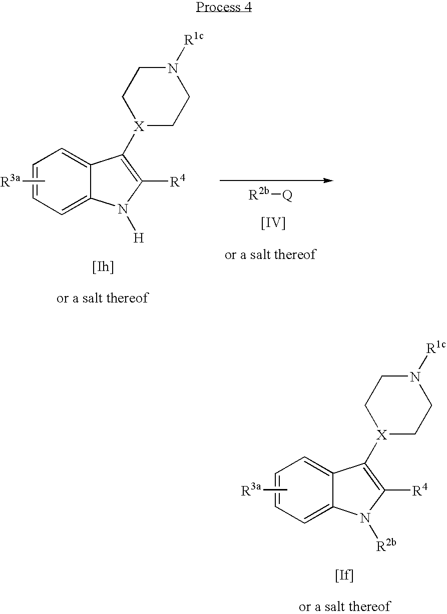
wherein
R^{1c} R^{2b}, R^{3a}, R⁴, X and Q are each as defined above.

The compound [If] or a salt thereof can be prepared by reacting a compound [Ih] or a salt thereof with a compound [IV] or a salt thereof.

Suitable salts of the compound [IV] may be the same as those exemplified for the compound [I].

The reaction can be carried out in substantially the same manner as Process 1, and therefore the reaction mode and reaction condition of this reaction are to be referred to those explained in Process 1.
Process 5 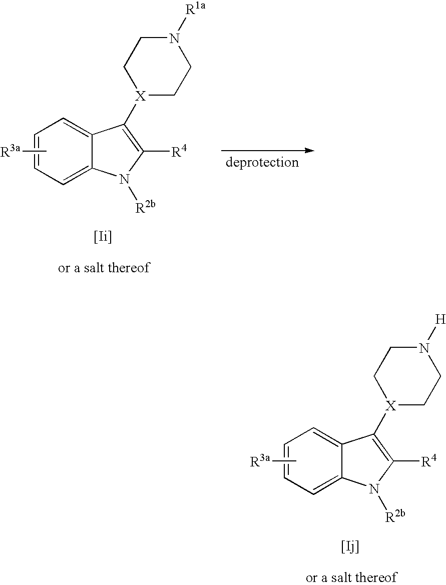
wherein
R^{1a}, R^{2b}, R^{3a}, R⁴ and X are each as defined above.

The compound [Ij] or a salt thereof can be prepared by subjecting a compound [Ii] or a salt thereof to deprotection reaction.

The reaction can be carried out in substantially the same manner as Process 2, and therefore the reaction mode and reaction condition of this reaction are to be referred to those explained in Process 2. 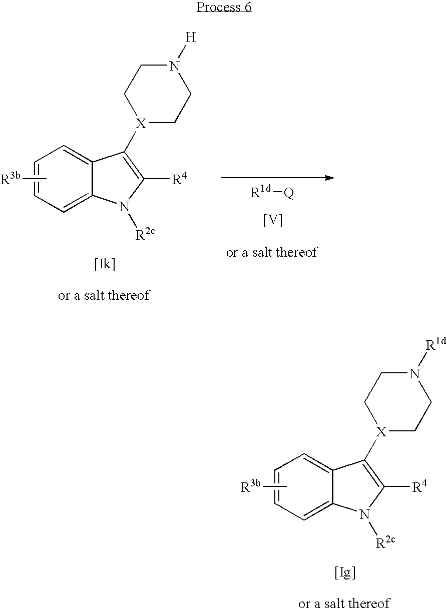
wherein
R^{1d}, R^{2c}, R^{3b}, R⁴, X and Q are each as defined above.

The compound [Ig] or a salt thereof can be prepared by reacting a compound [Ik] or a salt thereof with a compound [V] or a salt thereof.

Suitable salts of the compound [V] may be the same as those exemplified for the compound [I].

The reaction can be carried out in substantially the same manner as Process 1, and therefore the reaction mode and reaction condition of this reaction are to be referred to those explained in Process 1.

The compounds [I] and [Ia] to [Ik] and the starting compounds thereof can also be prepared by the methods of Examples mentioned below or similar manners thereto or conventional manners.

The compounds obtained by the above processes can be isolated and purified by a conventional method such as pulverization, recrystallization, chromatography, reprecipitation, or the like.

It is to be noted that the compounds of formula [Ia] to [Ik] are included within the scope of the compound of formula [I], and accordingly, in the above and subsequent description of the present specification and claims, description or suitable examples as to the compound [I] can be applied to the compounds [Ia] to [Ik].

The indole compounds represented by the formula [I] or its salts thereof possess potentiation of TGF-beta activity, therefore are useful for preventing and/or treating TGF-beta mediated diseases, especially bone diseases in human beings or animals.

Therefore, the compound [I] or its salt is useful for preventing and/or treating bone diseases such as low bone mass, osteoporosis, bone fracture, bone refracture, bone defect, osteomalacia, Behcet's syndrome in bone, osteotomy, cartilage defect, Paget's disease, rigid myelitis, chronic rheumatoid arthritis, chronic rheumatoid arthritis involving a cartilage, osteoarthritis (e.g., osteoarthritis of the knee), osteoarthritis involving cartilage (e.g., osteoarthritis of a knee involving cartilage), bone loss associated with periodontitis, prosthetic ingrowth, alveolar or mandibular bone loss, childhood idiopathic bone loss or secondary osteoporosis which includes glucocorticoid-induced osteoporosis, hyperthyroidism-induced osteoporosis, immobilization-induced osteoporosis, heparin-induced osteoporosis or immuno-suppressive-induced osteoporosis.

Further, the compound [I] or its salt may be useful for other diseases treatable by increasing the level of TGF-beta such as ocular diseases such as cataracts and glaucoma, cancer and its metastasis, infections by viruses such as HIV and HTLV 1 and 2 (human immunodeficiency virus and human T-cell lymphocyte virus) and the consequences thereof such as ATL (Adult T-cell Leukemia), leukemia, myelopathies and arthropathies, AIDS, immune deficiencies, autoimmune disorders such as multiple sclerosis, Sjogren's syndrome, Crohn's disease, and immune-related glomerulonephritis, neurodegenerative disorders such as Alzheimer's disease and Parkinson's disease, cell aging, tissue degeneration phenomena, inflammations such as acute or chronic rheumatoid arthritis and asthma, cell proliferation, graft rejection, diabetes such as type I diabetes or type II diabetes, hyperlipidemia, hyperinsulinism, hypertension, myelodysplasic syndrome such as aplastic anemia, ARDS(adult respiratory distress syndrome), prostatic hypertrophy, atherosclerosis, liver diseases such as hepatitis (e.g C,A,B,F) and liver cancer, septic shock, cachexia, renal diseases such as glomerulonephritis, ischemic pathologies such as myocardial infarction, myocardial ischemia, angina and cardiac failure or chronic pancreatitis.

Further, it is expected that the compound [I] or its salt have less side effects than other TGF-beta receptor agonists since it can potentiate endogenous TGF-beta in the patients.

In order to show the utility of the compound [I], pharmacological data of the representative compounds thereof are shown in the following.

### Assay using Mouse Calvarial Organ Culture

Test Method

Calvaria bone formation assay was essentially performed as described by Bonewald et al. (Endocrinology 139:3178, 1998). The calvaria from 5-day-old ICR mice were excised and cut in half along the sagittal suture. Each half of the calvaria was placed on a stainless steel grid in a 12-well tissue culture dish. Each well contained BGJ media (Sigma) supplemented with 0.1% bovine serum albumine, to which the test compound was added in combination with TGF-beta1. The media was changed 24 and 96 hrs. Calvaria were maintained in humidified air (5% CO₂) at 37° C. for 1 week. The calvaria were then fixed overnight in formalin, decalcified in EDTA, and were then embedded in paraffin wax. Calvaria sections were stained with hematoxylin and eosin. Histomorphometric analysis was performed using the Image-pro Plus (Trade mark, Media Cybernetics). The sum of the new bone area was determined. Rate of increase was calculated in the following;

$$Rate of increase ( \% ) = \frac{Ats - Ac}{Atg - Ac} \times 100$$

Calvaria area is calculated using Histomorphometric analysis.
Ats: Score of area in case of addition of test compound and TGF beta1
Atg: Score of area in case of addition of TGF beta1
Ac: Score of area in case of no addition (Control)

Test Result:

| Test Compound | |
|---|---|
| Example No. | Rate of increase |
| (1.0 × 10⁻⁵ M) | (%) |
| 7 | 206 |
| 9-(5) | 180 |
| 9-(18) | 158 |
| 40-(3) | 140 |
| 46-(4) | 203 |
| 49-(6) | 112 |

The pharmaceutical composition of the present invention can be used in the form of a pharmaceutical preparation, for example, in solid, semisolid or liquid form (e.g., tablet, pellet, troche, capsule, suppository, cream, ointment, aerosol, powder, solution, emulsion, suspension, sponge carrier placed into fracture site etc.), which contains the compound [I] or a pharmaceutically acceptable salt thereof as an active ingredient, suitable for rectal, pulmonary (nasal or buccal inhalation), nasal, ocular, external (topical), oral or parenteral (including subcutaneous, intravenous and intramuscular) administrations or insulation.

The pharmaceutical composition of this invention can contain various organic or inorganic carrier materials, which are conventionally used for pharmaceutical purpose, such as excipient (e.g., sucrose, starch, mannit, sorbit, lactose, glucose, cellulose, talc, calcium phosphate, calcium carbonate, etc.), binding agent (e.g., cellulose, methyl cellulose, hydroxypropylcellulose, polypropylpyrrolidone, gelatin, gum arabic, polyethyleneglycol, sucrose, starch, etc.), disintegrator (e.g., starch, carboxymethylcellulose, calcium salt of carboxymethyl cellulose, hydroxypropylstarch, sodium glycol- starch, sodium bicarbonate, calcium phosphate, calcium citrate, etc.), lubricant (e.g., magnesium stearate, talc, sodium laurylsulfate, etc.), flavoring agent (e.g., citric acid, mentol, glycine, orange powders, etc.), preservative (e.g., sodium benzoate, sodium bisulfite, methylparaben, propylparaben, etc.), stabilizer (e.g., citric acid, sodium citrate, acetic acid, etc.), suspending agent (e.g., methyl cellulose, polyvinylpyrrolidone, aluminum stearate, etc.), dispersing agent, aqueous diluting agent (e.g., water), base wax (e.g., cacao butter, polyethyleneglycol, white petrolatum, etc.), solubilizing agent (e.g., sodium benzoate, potassium iodide, etc.), surfactants (e.g., sodium lauryl sulfate, polyoxyethylenehydrogenated castor oil, etc.).

The effective ingredient may usually be administered with a unit dose of 0.001 μg/an application site to 20 μg/an application site or 0.0001 mg/kg to 10 mg/kg, 1 to 4 times a day or 1 to 4 times a week.

However, the above dosage may be increased or decreased according to age, weight, conditions of the patient or the administering method.

The injection of the effective ingredient can be controlled by CT scanning or X-ray monitoring means.

Further, compound [I] or its salt may also be administrated or applied simultaneously, separately or sequentially with TGF-beta to human being or animals.

The following Examples are given only for the purpose of illustrating the present invention in more details.

Abbreviations and acronyms used in the Examples and full name thereof are described in the following.

| Abbreviations and | |
|---|---|
| Acronyms | Full Name |
| AcOEt or EtOAc | ethyl acetate |
| AcOH | acetic acid |
| BuOH, t-BuOH, etc. | butanol, t-butyl alcohol, etc. |
| DME | 1,2-dimethoxyethane |
| DMF | N,N-dimethylformamide |
| DMSO | dimethyl sulfoxide |
| Et3N | triethylamine |
| EtOH | ethanol |
| IPE | diisopropyl ether |
| MeOH | methanol |
| PrOH, i-PrOH, etc. | propanol, isopropyl alcohol, etc. |
| TFA | trifluoroacetic acid |
| THF | tetrahydrofuran |
| EDCI | 1-ethyl-3-[3′-(dimethylamino)propyl]carbodiimide |
| HOBt or HOBT | 1-hydroxybenztriazole |
| BSU | bis(trimethylsilyl)urea |
| MSA | mono(trimethylsilyl)acetamide |
| Pd/C | palladium on carbon |
| Deg | ° C. = degree centigrade |
| Min | minute(s) |
| hr or h | hour(s) |
| conc. | concentrated |
| Aq | aqueous (ex. aq NaHCO3 solution) |

### EXAMPLE 1

DMF (500 ml), 3-(4-piperidinyl)-1H-indole (50 g) and triethylamine (45.5 ml) were combined. A solution of di-tert-butyl dicarbonate (71 g) in methylene chloride (70 ml) was dropwise added to the reaction mixture in ice bath. After that, the reaction mixture was stirred for 2.5 h. The mixture was poured into ice water (1500 ml) and the mixture was stirred for 1 h. Sodium chloride was added to the mixture and the mixture was stirred for 30 min. The crystalline precipitate was collected by filtration and washed with water and the solution of IPE and hexane (1:1). The residue was dried in vacuo to give tert-butyl 4-(1H-indol-3-yl)-1-piperidinecarboxylate (74.32 g).

tert-butyl 4-(1H-indol-3-yl)-1-piperidinecarboxylate (74.32 g).

mp: 159-160° C.

### EXAMPLE 2

tert-Butyl 4-(1H-indol-3-yl)-1-piperidinecarboxylate (1.07 g) was dissolved in DMF (10 ml). Sodium hydride (60%, 160 mg) was added to the mixture at 0° C. The mixture was stirred at room temperature for 30 min. The mixture was cooled with ice bath and a solution of 4-methylsulfonylbenzyl bromide (887 mg) in DMF (5 ml) was added to the mixture. The reaction mixture was stirred at room temperature for 1 h, and then poured into ice water. The mixture was extracted with AcOEt and the organic layer was washed with water and brine, and then dried over sodium sulfate. The organic layer was evaporated in vacuo. The residue was purified with silica gel chromatography (AcOEt/Hexane=1/10 to 1/3 elution) to give tert-butyl 4-[1-[4-(methylsulfonyl)benzyl]-1H-indol-3-yl]-1-piperidinecarboxylate (711 mg).

Mass: m/z 469(M+H)⁺

### EXAMPLE 3

The following compounds were obtained according to a similar manner to that of Example 2.

| Ex- | | Mass(m/z) or |
|---|---|---|
| ample | Chemical structure | ¹H-NMR(δ) |
| 3-(1) | 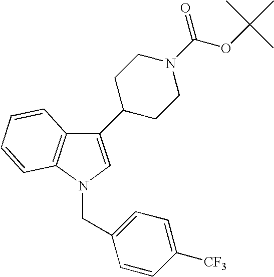 | 359 (M − Boc + H)⁺ |
| 3-(2) | 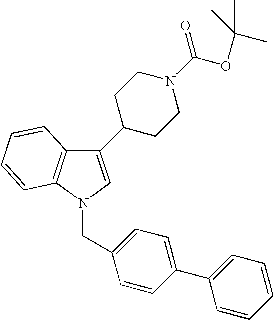 | (Solvent: DMSO-d₆) 7.55-7.60(5H, m), 7.37-7.43(3H, m), 7.24-7.34(4H, m), 6.95-7.10(2H, m), 5.35(2H, s), 4.05-4.08(2H, m), 2.85-2.98(3H, m), 1.92-1.97(2H, m), 1.45-1.55(2H, m), 1.38(9H, s) |
| 3-(3) | 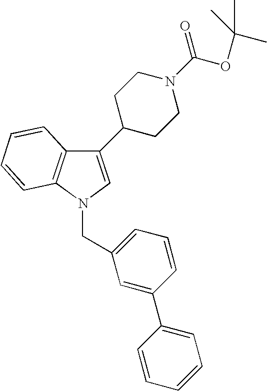 | N.D |
| 3-(4) | 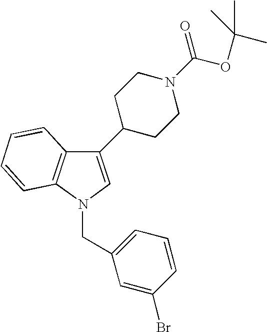 | (Solvent: DMSO-d₆) 7.56(1H, d, J=8Hz), 7.42-7.35(3H, m), 6.98-7.23(5H, m), 5.32(2H, s), 4.00-4.05(2H, m), 2.87-2.95(3H, m), 1.90-1.95(2H, m), 1.43-1.52(2H, m), 1.38(9H, s) |
| 3-(5) | 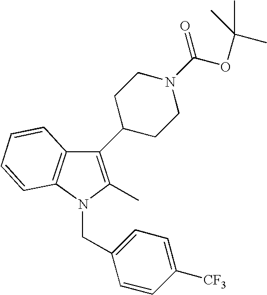 | 473(M + H)⁺ |
| 3-(6) | 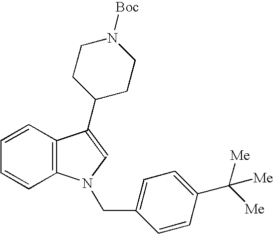 | 447(M + H)⁺ |
| 3-(7) | 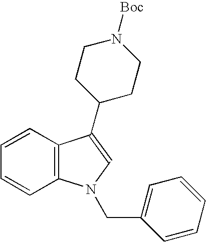 | 291(M − Boc +H)⁺ |
| 3-(8) | 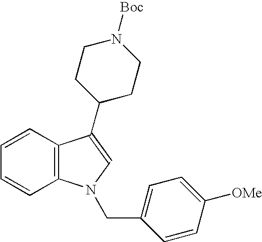 | 321(M − Boc +H)⁺ |
| 3-(9) | 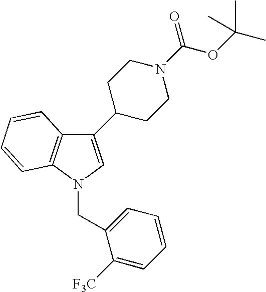 | N.D |

### EXAMPLE 4

tert-Butyl 4-(1H-indol-3-yl)-1-piperidinecarboxylate (17.5 g), benzene (290 ml), tetrabutylammonium hydrogensulfate (2.0 g) and 50% aqueous solution of sodium hydroxide were combined. A solution of methanesulfonyl chloride (6.8 ml) in benzene (100 ml) was added to the mixture at room temperature. After the reaction mixture was stirred for 1.5 h, methanesulfonyl chloride (1.3 ml) was added to the mixture. Further, methanesulfonyl chloride (1.0 ml) was added half an hour later. After 30 minutes, 50% aqueous sodium hydroxide was added to the mixture and the mixture was stirred for 2 h. The mixture was filtered and the filtrate was washed with water. The filtrate was dried over sodium sulfate and evaporated in vacuo. The residue was collected by filtration and washed with IPE to give tert-butyl 4-[1-(methylsulfonyl)-1H-indol-3-yl]-1-piperidinecarboxylate (15.2 g).

mp: 136-137° C.

### EXAMPLE 5

tert-Butyl 4-(1H-indol-3-yl)-1-piperidinecarboxylate (1.00 g), 4-(trifluoromethyl)benzoyl chloride (989 μl), triethylamine (928μl), 4-dimethylaminopyridine (41 mg) and methylene chloride (20 ml) were combined. The mixture was refluxed for 3 days. The reaction mixture was cooled with ice bath and N,N′-diethyl-1,3-propanediamine (525 μl) was added to the mixture. The mixture was stirred for 20 min and methylene chloride was added to the mixture. The mixture washed with water, 1N-hydrochloric acid, water, and brine, and then dried over magnesium sulfate. The organic layer was evaporated in vacuo. The residue was purified with silica gel chromatography (toluene/AcOEt=1/0 to 5/1 elution) to give tert-butyl 4-[1-[4-(trifluoromethyl)benzoyl]-1H-indol-3-yl]-1-piperidinecarboxylate (1.41 g).

Mass: m/z 373(M-Boc+H )⁺

### EXAMPLE 6-(1)

3-(1-Acetyl-4-piperidinyl)-1-(4-nitrobenzyl)indoline (1.61 g) was added to 47% hydrobromic acid (45 ml) and the mixture was heated at 100° C. for 24 h. The mixture was evaporated in vacuo and water was added to the residue. The mixture was basified with 15% aqueous solution of sodium hydroxide and extracted with methylene chloride. The organic layer was washed with brine and dried over magnesium sulfate. The organic layer was evaporated in vacuo and the residue was dissolved in methylene chloride (30 ml). Triethylamine (1.34 ml) and di-tert-butyl dicarbonate (1.61 g) were added to the mixture and the mixture was stirred for 2 h. Methylene chloride was added to the mixture and the mixture was washed with water, 1N-hydrochloric acid, water, saturated sodium bicarbonate solution, water, and brine in order. The organic layer was dried over magnesium sulfate and evaporated in vacuo. The residue was purified with silica gel chromatography (hexane/AcOEt=6/1 to 4/1 elution) to give tert-butyl 4-[1-(4-nitrobenzyl)-1H-indolin-3-yl]-1-piperidinecarboxylate (1.41 g).

Mass: m/z 338(M-Boc+H)⁺

### EXAMPLE 6-(2)

tert-Butyl 4-[1-(4-nitrobenzyl)-1H-indolin-3-yl]-1-piperidinecarboxylate (1.41 g), manganese(IV) oxide (1.04 g) and nitrobenzene (20 ml) were combined and heated at 100° C. for 5 minutes. The reaction mixture was cooled to room temperature and filtered. The filtrate was evaporated in vacuo and the residue was purified with silica gel chromatography (hexane/AcOEt=4/1 to 2/1 elution) to give tert-butyl 4-[1-(4-nitrobenzyl)-1H-indol-3-yl]-1-piperidinecarboxylate (0.77 g).

Mass: m/z 336(M-Boc+H )⁺

### EXAMPLE 7

tert-Butyl 4-[1-[4-(methylsulfonyl)benzyl]-1H-indol-3-yl]-1-piperidinecarboxylate (686 mg) was dissolved in AcOEt (10 ml). To the solution, 4N-hydrogen chloride in AcOEt (4 ml) was added. The mixture was stirred for 1 h. The precipitate was collected by filtration and washed with AcOEt and ether to give 1-[4-(methylsulfonyl)benzyl]-3-(4-piperidinyl)-1H-indole hydrochloride (525 mg).

Mass: m/z 369 (M-HCl+H)⁺

### EXAMPLE 8

The following compounds were obtained according to a similar manner to that of Example 7.

| Ex- | | Mass(m/z) or |
|---|---|---|
| ample | Chemical structure | IR(cm⁻¹) |
| 8-(1) | 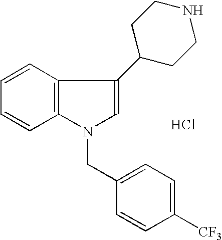 | 359 (M − HCl + H)⁺ |
| 8-(2) | 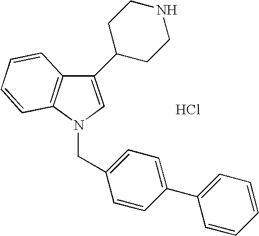 | 367 (M − HCl + H)⁺ |
| 8-(3) | 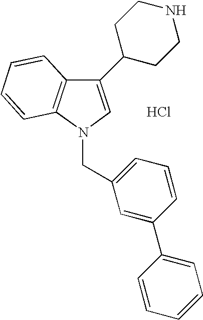 | 367 (M − HCl + H)⁺ |
| 8-(4) | 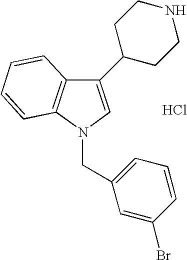 | 369 (M − HCl)⁺371 (M − HCl + 2H)⁺ |
| 8-(5) | 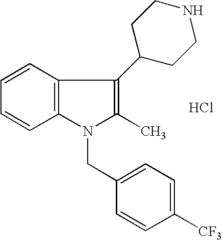 | 373 (M − HCl + H)⁺ |
| 8-(6) | 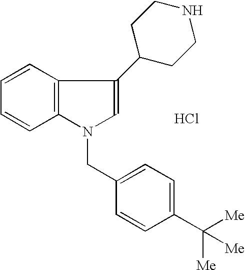 | 347 (M − HCl + H)⁺ |
| 8-(7) | 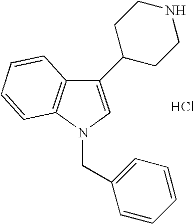 | 291 (M − HCl + H)⁺ |
| 8-(8) | 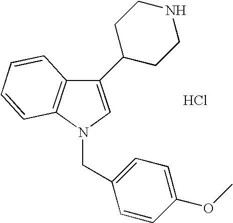 | 321 (M − HCl + H)⁺ |
| 8-(9) | 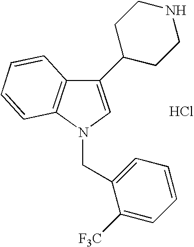 | 359 (M − HCl + H)⁺ |
| 8-(10) | 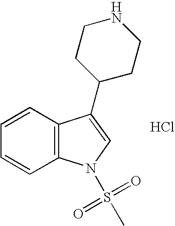 | IR(cm⁻¹) 1350, 1100 |
| 8-(11) | 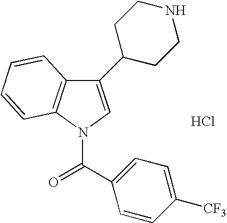 | 373 (M − HCl + H)⁺ |
| 8-(12) | 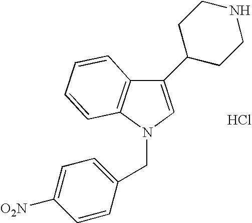 | 336 (M − HCl + H)⁺ |

### EXAMPLE 9

The following compounds were obtained according to a similar manner to that of Example 7, from the starting compounds each of which was obtained according to a similar manner to that of Example 2 by reacting tert-butyl 4-(1H-indol-3-yl)-1-piperidinecarboxylate with a corresponding ar(lower)alkyl halide compound or a corresponding heteroarylalkyl halide compound.

| Ex- | | |
|---|---|---|
| am- | | |
| ple | Chemical structure | Mass(m/z) |
| 9-(1) | 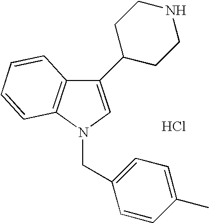 | 305 (M −HCl + H)⁺ |
| 9-(2) | 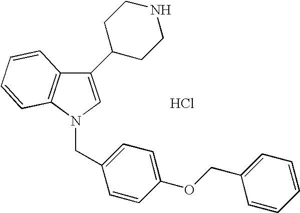 | 397 (M −HCl + H)⁺ |
| 9-(3) | 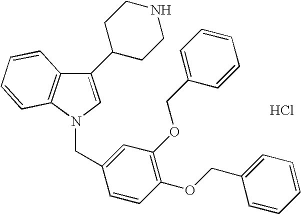 | 503 (M −HCl + H)⁺ |
| 9-(4) | 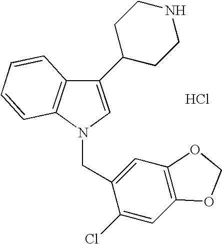 | 369 (M −HCl + H)⁺ |
| 9-(5) | 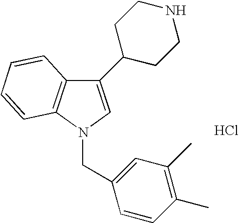 | 319 (M −HCl + H)⁺ |
| 9-(6) | 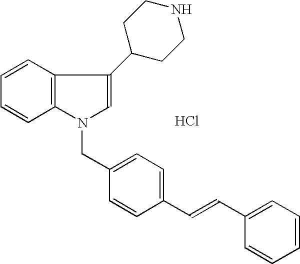 | 393 (M −HCl + H)⁺ |
| 9-(7) | 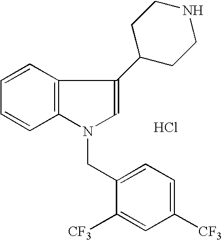 | 427 (M −HCl + H)⁺ |
| 9-(8) | 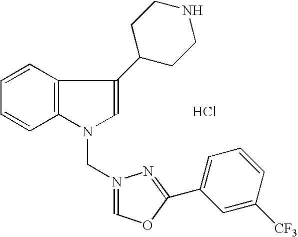 | 427 (M −HCl + H)⁺ |
| 9-(9) | 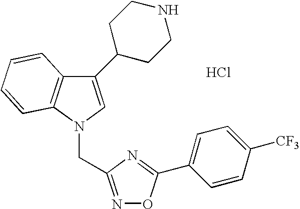 | 427 (M −HCl + H)⁺ |
| 9- (10) | 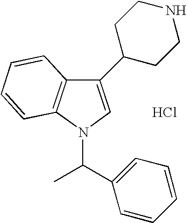 | 305 (M −HCl + H)⁺ |
| 9- (11) | 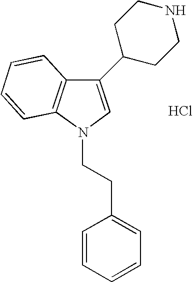 | 305 (M −HCl + H)⁺ |
| 9- (12) | 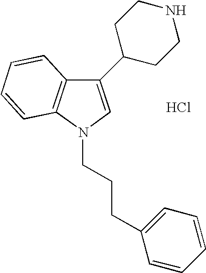 | 319 (M −HCl + H)⁺ |
| 9- (13) | 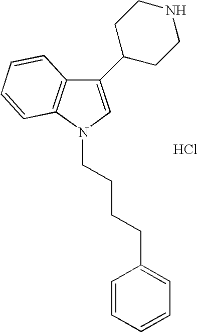 | 333 (M −HCl + H)⁺ |
| 9- (14) | 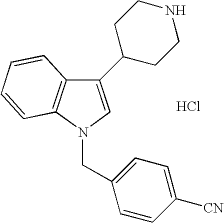 | 316 (M −HCl + H)⁺ |
| 9- (15) | 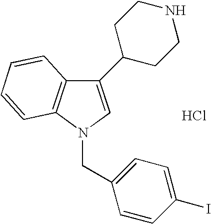 | 417 (M −HCl + H)⁺ |
| 9- (16) | 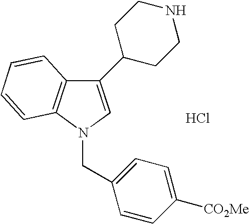 | 349 (M −HCl + H)⁺ |
| 9- (17) | 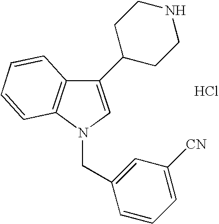 | 316 (M −HCl + H)⁺ |
| 9- (18) | 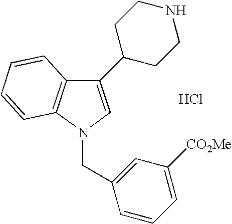 | 349 (M −HCl + H)⁺ |
| 9- (19) | 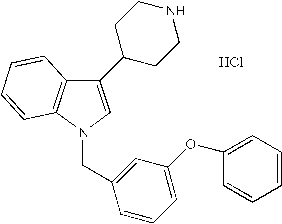 | 383 (M −HCl + H)⁺ |
| 9- (20) | 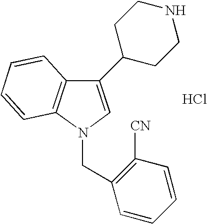 | 316 (M −HCl + H)⁺ |
| 9- (21) | 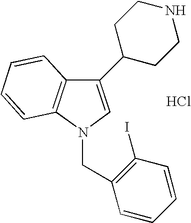 | 417 (M −HCl + H)⁺ |
| 9- (22) | 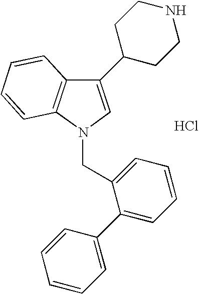 | 367 (M −HCl + H)⁺ |
| 9- (23) | 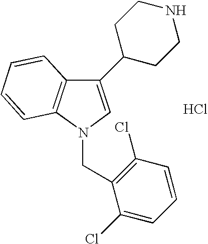 | 359(M − HCl)⁺361 (M −HCl + 2H)⁺ |
| 9- (24) | 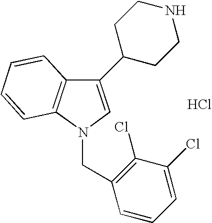 | 359(M − HCl)⁺361 (M −HCl + 2H)⁺ |
| 9- (25) | 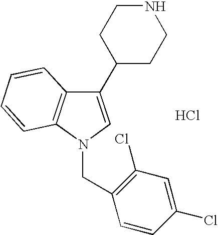 | 359(M − HCl)⁺361 (M −HCl + 2H)⁺ |
| 9- (26) | 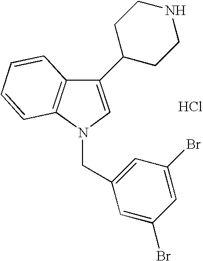 | 449 (M −HCl + H)⁺ |
| 9- (27) | 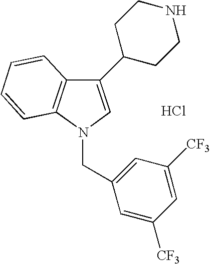 | 427 (M −HCl + H)⁺ |
| 9- (28) | 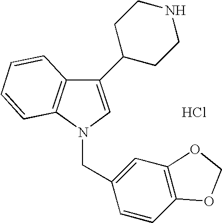 | 335 (M −HCl + H)⁺ |
| 9- (29) | 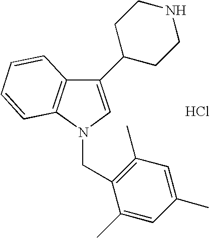 | 333 (M −HCl + H)⁺ |
| 9- (30) | 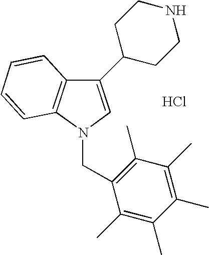 | 361 (M −HCl + H)⁺ |
| 9- (31) | 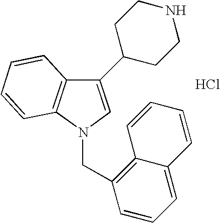 | 341 (M −HCl + H)⁺ |
| 9- (32) | 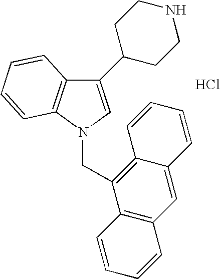 | 391 (M −HCl + H)⁺ |
| 9- (33) | 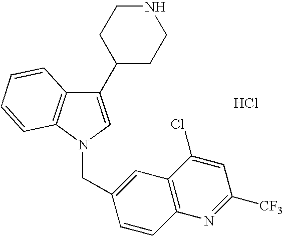 | 444 (M −HCl + H)⁺ |
| 9- (34) | 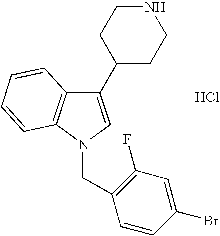 | 387(M − HCl)⁺389 (M −HCl + 2H)⁺ |
| 9- (35) | 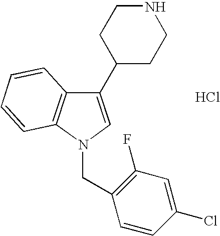 | 343 (M −HCl + H)⁺ |
| 9- (36) | 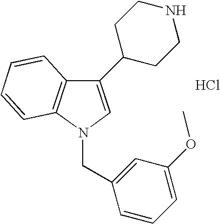 | 321 (M −HCl + H)⁺ |
| 9- (37) | 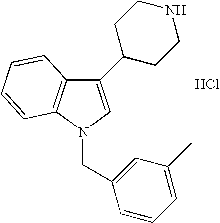 | 305 (M −HCl + H)⁺ |
| 9- (38) | 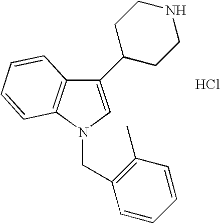 | 305 (M −HCl + H)⁺ |
| 9- (39) | 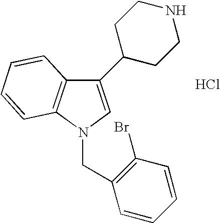 | 369(M − HCl)⁺371 (M −HCl + 2H)⁺ |
| 9- (40) | 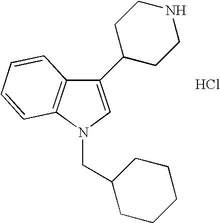 | 297 (M −HCl + H)⁺ |
| 9- (41) | 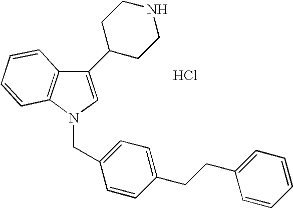 | 395 (M −HCl + H)⁺ |
| 9- (42) | 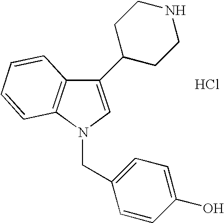 | 307 (M −HCl + H)⁺ |
| 9- (43) | 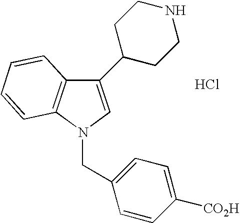 | 335 (M −HCl + H)⁺ |
| 9- (44) | 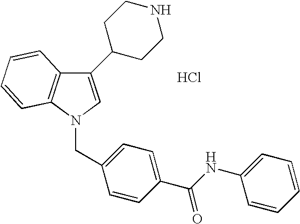 | 410 (M −HCl + H)⁺ |
| 9- (45) | 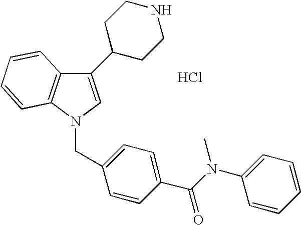 | 424 (M −HCl + H)⁺ |

### EXAMPLE 10

1-(Phenylsulfonyl)-3-(4-piperidinyl)-1H-indole hydrochloride was obtained according to a similar manner to that of Example 7 by using tert-butyl 4-{1-(phenylsulfonyl)-1H-indol-3-yl}-1-piperidinecarboxylate obtained according to a similar manner to that of Example 4 by reacting tert-butyl 4-(1H-indol-3-yl)-1-piperidinecarboxylate with phenylsulfonyl halide.

Mass: m/z 341 (M-HCl+H)⁺

### EXAMPLE 11

4-[[3-(1-Acetyl-4-piperidinyl)-1H-indol-1-yl]methyl]aniline (170 mg), ETOH (5 ml), and 1N-aqueous sodium hydroxide (5 ml) were combined and the mixture was refluxed for 2.5 h. The mixture was cooled and water was added to the mixture. The mixture was extracted with a mixture of methylene chloride and methanol (20:1) and the organic layer was washed with brine. The organic layer was dried over magnesium sulfate, and evaporated in vacuo. The residue was dissolved with EtOH and 4N-solution of hydrogen chloride in dioxane (0.5 ml) was added therto. The mixture was evaporated in vacuo and the residue was crystallized with a mixture of EtOH and IPE. The crystal was collected by filtration and washed with EtOH and IPE to give 4-[[3-(4-piperidinyl)-1H-indol-1-yl]methyl]aniline dihydrochloride (141 mg).

Mass: m/z 306 (M-2HCl+H)⁺

### EXAMPLE 12-(1)

3-(1-Acetyl-4-piperidinyl)-1H-indole (48.2 g) was added to AcOH (1000 ml) and the mixture was stirred at 15-20° C. Sodium cyanoborohydride (105 g) was added to the mixture for 1.5 h. Then, the mixture was stirred for 4 h. Water (500 ml) was added to the mixture and the mixture was evaporated in vacuo. 2N aqueous solution of sodium hydroxide (1500 ml) was added to the residue and the mixture was extracted with AcOEt. The organic layer was washed with brine and dried over magnesium sulfate. The organic layer was evaporated in vacuo, and the residue was purified with silica gel chromatography (chloroform/methanol=50/1 elution) to give 3-(1-acetyl-4-piperidinyl)-1H-indoline (41.1 g).

EXAMPLE 12-(2)

3-(1-Acetyl-4-piperidinyl)-1H-indoline (2.51 g), 4-nitrobenzyl bromide (2.22 g), potassium carbonate (1.42 g), and DMF (25 ml) was combined and the mixture was stirred for 22 h. Water was added to the reaction mixture and extracted with toluene. The organic layer was washed with brine and dried over magnesium sulfate. The organic layer was evaporated in vacuo and the residue was purified with silica gel chromatography (hexane/AcOEt=1/1 to 1/9 elution) to give 3-(1-acetyl-4-piperidinyl)-1-(4-nitrobenzyl)indoline (2.43 g).

Mass: m/z 380(M+H)⁺

### EXAMPLE 12-(3)

3-(1-Acetyl-4-piperidinyl)-1-(4-nitrobenzyl)indoline (1.24 g), manganese(IV) oxide (1.66 g) and nitrobenzene (12.4 ml) were combined and the mixture was heated at 150° C. for 1.5 h. The reaction mixture was cooled to room temperature and filtered. The filtrate was evaporated in vacuo and the residue was purified with silica gel chromatography (methylene chloride/ methanol=100/1 to 15/1 elution) to give 3-(1-acetyl-4-piperidinyl)-1-(4-nitrobenzyl)-1H-indole (1.10 g).

Mass: m/z 378(M+H)⁺

### EXAMPLE 13

3-(1-Acetyl-4-piperidinyl)-1-(4-nitrobenzyl)-1H-indole (0.35 g), ammonium formate (0.3 g), ethanol (7 ml), water (0.7 ml) and 10% palladium on carbon (70 mg) were combined under hydrogen atmosphere (1atm). The reaction mixture was stirred at 50° C. for 3.5 h then filtered. The filtrate was evaporated in vacuo and the residue was purified with silica gel chromatography (methylene chloride/methanol=50/1 to 25/1 elution) to give 4-[[3-(1-acetyl-4-piperidinyl)-1H-indol-1-yl]methyl]aniline (0.25 g).

Mass: m/z 348(M+H)⁺

### EXAMPLE 14

3-(4-Piperidinyl)-1-[4-(trifluoromethyl)benzyl]-1H-indole hydrochloride (0.40 g), 3-phthalimidopropyl bromide (0.27 g), sodium hydrogencarbonate (0.18 g) and DMF (10 ml) were combined and the mixture was heated at 70° C. for 8 h. After cooled, the reaction mixture was added to water (50 ml) and the mixture was extracted with methylene chloride. The organic layer was washed with water and brine and dried over magnesium sulfate. The organic layer was evaporated in vacuo and the residue was purified with silica gel chromatography (methylene chloride/methanol=50/1 to 10/1 elution) to give 3-[1-(3-phthalimidopropyl)-4-piperidinyl]-1-[4-(trifluoromethyl)benzyl]-1H-indole (0.414 g).

Mass : m/z 546(M+H)⁺

### EXAMPLE 15

3-[1-(6-Phthalimidohexyl)-4-piperidinyl]-1-[4-(trifluoromethyl)benzyl]-1H-indole was obtained according to a similar manner to that of Example 14.

Mass: m/z 588(M+H)⁺

### EXAMPLE 16

3-[1-(3-Phthalimidopropyl)-4-piperidinyl]-1-[4-(trifluoromethyl)benzyl]-1H-indole (200 mg), hydrazine monohydrate (90 μl) and THF (5 ml) were combined and refluxed for 8 h. After cooled, the mixture was filtered and the filtrate was evaporated in vacuo. The residue (0.17 g) was dissolved in methylene chloride (5 ml) and triethylamine (51 μl) and di-tert-butyl dicarbonate (80 mg) were added to the mixture in order and the mixture was stirred for 4 h. The mixture was evaporated in vacuo and AcOEt was added to the residue. The organic layer was washed with saturated sodium bicarbonate solution, water and brine in order and dried over magnesium sulfate. The organic layer was evaporated in vacuo and the residue was purified with silica gel chromatography (methylene chloride/methanol=50/1 to 25/2 elution) to give 3-[1-(3-tert-butoxycarbonylaminopropyl)-4-piperidinyl-1-[4-(trifluoromethyl)benzyl]-1H-indole (1 16 mg).

Mass: m/z 516(M+H)⁺

### EXAMPLE 17

3-[1-(6-tert-Butoxycarbonylaminohexyl)-4-piperidinyl]-1-[4-(trifluoromethyl) benzyl]-1H-indole was obtained according to a similar manner to that of Example 16.

Mass: m/z 558(M+H)⁺

### EXAMPLE 18-(1)

A solution of 7-[(tert-butoxycarbonyl)amino]heptanoic acid (25 g) in THF(1500 ml) was cooled to 0° C. under nitrogen atmosphere. Borane-dimethyl sulfide complex (29 ml) was added to the solution. The mixture was stirred at 0° C. for 2 h. 1N-aqueous solution of sodium hydroxide (326 ml) was added to the mixture at 5-10° C. over 1 h. The mixture was stirred at room temperature for 1 h. Then THF was removed in vacuo. The remaining aqueous solution was extracted with Et2O. The combined organic layer was washed with brine. The organic layer was dried over magnesium sulfate, and evaporated in vacuo to give crude colorless oil (24.1 g). The crude oil was dissolved in THF (500 ml) under nitrogen atmosphere. Triphenylphosphine (34.7 g) and carbon tetrabromide (43.9 g) were added to the solution at room temperature, and the mixture was stirred at room temperature for 13 h. The reaction mixture was filtered, and the filtrate was evapolated in vacuo. The residue was purified with silica gel chromatography (AcOEt/hexane=1/10 to 1/4 elution) to give tert-butyl (7-bromoheptyl)carbamate (22.6 g).

mp. 48-49° C.

### EXAMPLE 18-(2)

3-[1-(7-tert-Butoxycarbonylaminohepthyl)-4-piperidinyl]-1-[4-(trifluoromethyl) benzyl]-1H-indole was obtained according to a similar manner to that of Example 14 by using tert-butyl(7-bromoheptyl)carbamate and 3-(4-piperidinyl)-1-[4-(trifluoromethyl)benzyl]-1H-indole hydrochloride.

Mass: m/z 572(M+H)⁺

### EXAMPLE 19

The following compounds were obtained according to a similar manner to that of Example 7.

| Example | Chemical structure | Mass(m/z) |
|---|---|---|
| 19-(1) | 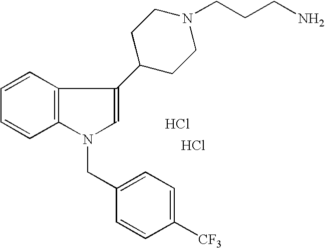 | 416(M − 2HCl + H)⁺ |
| 19-(2) | 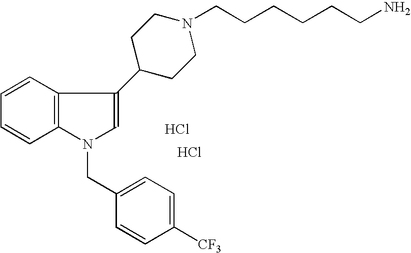 | 458(M − 2HCl + H)⁺ |
| 19-(3) | 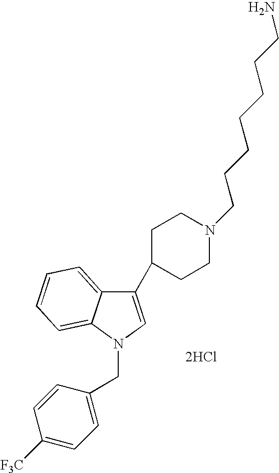 | 472(M − 2HCl + H)⁺ |

### EXAMPLE 20

3-(4-Piperidinyl)-1-[4-(trifluoromethyl)benzyl]-1H-indole hydrochloride (300 mg), iodomethane (5.2 μl), sodium hydrogencarbonate (134 mg) and DMF (6 ml) were combined and the mixture was stirred for 24 h. Water was added to the mixture and the mixture was extracted with methylene chloride. The organic layer was washed with brine and dried over magnesium sulfate. The organic layer was evaporated in vacuo and the residue was purified with silica gel chromatography (methylene chloride/methanol=50/1 to 10/1 elution). The desired fraction was evaporated in vacuo and the residue was dissolved in AcOEt. The organic layer was washed with 1N-aqueous solution of sodium hydroxide, water and brine in order and dried over magnesium sulfate. The organic layer was evaporated in vacuo and the residue was dissolved in EtOH. 4N-solution of hydrogen chloride in dioxane (0.2 ml) was added to the solution and the solution was evaporated in vacuo to give 3-(1-methyl-4-piperidinyl)-1-[4-(trifluoromethyl)benzyl]-1H-indole hydrochloride (72 mg).

Mass: m/z 373 (M-HCl+H)⁺

### EXAMPLE 21

The following compounds were obtained according to a similar manner to that of Example 20.

| Ex- | | |
|---|---|---|
| am- | | |
| ple | Chemical structure | Mass(m/z) |
| 21- (1) | 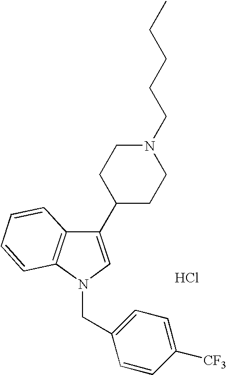 | 429 (M −HCl + H)⁺ |
| 21- (2) | 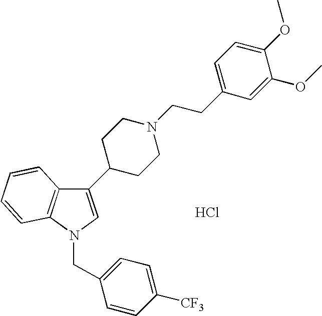 | 523 (M −HCl + H)⁺ |

### EXAMPLE 22

tert-Butyl 4-(1-methyl-1H-indol-3-yl)-1-piperidinecarboxylate was obtained according to a similar manner to that of Example 2 by reacting iodomethane with tert-buthyl 4-(1H-indol-3-yl)-1-piperidinecarboxylate.

Mass: m/z 215(M-Boc+H)⁺

### EXAMPLE 23

1-Methyl-3-(4-piperidinyl)-1H-indole hydrochloride was obtained according to a similar manner to that of Example 7.

Mass: m/z 215(M-HCl+H)⁺

### EXAMPLE 24

1-Methyl-3-(4-piperidinyl)-1H-indole hydrochloride (502 mg), 7-[(tert-butoxycarbonyl)-amino]heptanoic acid (491 mg), 1-hydroxybenzotriazole hydrate (270 mg) and methylene chloride (20 ml) were combined. To the mixture, 1-(3-dimethylaminopropyl)-3-ethylcarbodiimide (365 μl) was added and the mixture was stirred for 6 h. Methylene chloride was added to the mixture and the mixture was washed with water, saturated sodium bicarbonate solution, water, 1N-hydrochloric acid, water and brine in order. The organic layer was dried over magnesium sulfate and evaporated in vacuo to give 3-[1-(7-tert-butoxycarbonylaminoheptanoyl)-4-piperidinyl]-1-methyl-1H-indole (0.84 g).

Mass: m/z 442(M+H)⁺

### EXAMPLE 25

3-[1-(7-Aminoheptanoyl)-4-piperidinyl]-1-mehyl-1H-indole-hydrochloride was obtained according to a similar manner to that of Example 7.

Mass: m/z 342(M-HCl+H)⁺

### EXAMPLE 26

3-[1-(7-Aminoheptanoyl)-4-piperidinyl]-1-mehyl-1H-indole hydrochloride (0.70 g) was dissolved in diluted aqueous solution of sodium bicarbonate and the solution was extracted with a mixture of methylene chloride and methanol(10:1). The organic layer was washed with brine and dried over magnesium sulfate. The organic layer was evaporated in vacuo and azeotroped with toluene. Lithium aluminum hydride (0.14 g) and THF (14 ml) were combined under nitrogen atmosphere. The solution of the residue described above in THF (5 ml) was added to the mixture and the reaction mixture was stirred for 1.5 h. Water (0.14 ml), 15% solution of sodium hydroxide (0.14 ml) and water (0.42 ml) were added to the mixture in order and the mixture was filtered. The filtrate was evaporated in vacuo and the residue was dissolved in AcOEt. The solution was washed with brine and dried over magnesium sulfate. The organic layer was evaporated in vacuo and the residue was dissolved in THF (20 ml). To the solution, di-tert-butyl dicarbonate (0.40 g) was added and the mixture was stirred for 14 h. The mixture was diluted with methylene chloride and washed with water, saturated sodium bicarbonate solution, water and brine in order. The organic layer was dried over magnesium sulfate and evaporated in vacuo. The residue was purified with silica gel chromatography (methylene chloride/methanol=50/1 to 4/1 elution) to give 3-[1-(7-tert-butoxycarbonylaminohepthyl)]-4-piperidinyl-1-methyl-1H-indole (0.39 g).

Mass: m/z 428(M+H)⁺

### EXAMPLE 27

3-[1-(7-Aminoheptyl)-4-piperidinyl]-1-methyl-1H-indole dihydrochloride was obtained according to a similar manner to that of Example 7.

Mass: m/z 328(M-2HCl+H)⁺

### EXAMPLE 28

The following compounds were obtained according to a similar manner to that of Example 14.

| Example | Chemical structure | Mass(m/z) |
|---|---|---|
| 28-(1) | 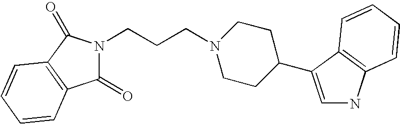 | N.D |
| 28-(2) | 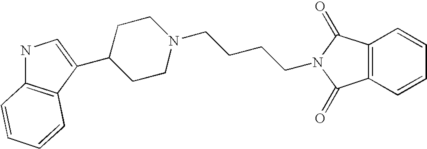 | 402(M + H)⁺ |
| 28-(3) | 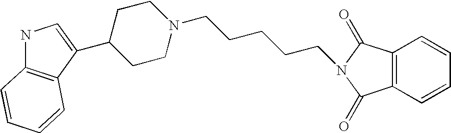 | 416(M + H)⁺ |
| 28-(4) | 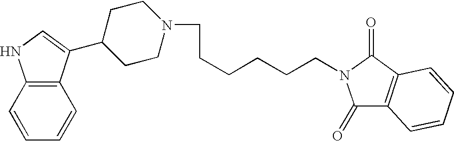 | 430(M + H)⁺ |
| 28-(5) | 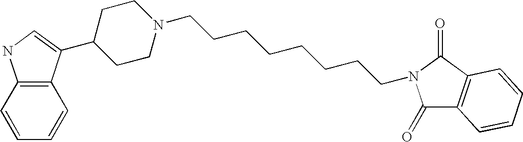 | 458(M + H)⁺ |

### EXAMPLE 29

3-[1-(3-Phthalimidopropyl)-4-piperidinyl]-1H-indole (5.86 g), hydrazine monohydrate (1.97 g) and EtOH(147 ml) were combined and refluxed for 1 h. After cooled, the mixture was filtered and the filtrate was evaporated in vacuo. To the residue, 5% solution of sodium hydroxide (280 ml) was added and the mixture was extracted with AcOEt. The organic layer was washed with water and brine and dried over magnesium sulfate. The organic layer was evaporated in vacuo to give 3-[1-(3-aminopropyl)-4-piperidinyl]-1H-indole (1.58 g).

### EXAMPLE 30

3-[1-(4-Aminobutyl)-4-piperidinyl]-1H-indole was obtained according to a similar manner to that of Example 29.

Mass: m/z 272(M+H)⁺

### EXAMPLE 31

3-[1-(7-tert-Butoxycarbonylaminoheptanoyl)-4-piperidinyl]-1H-indole was obtained according to a similar manner to that of Example 24.

Mass: m/z 428(M+H)⁺

### EXAMPLE 32

3-[1-(7-Aminoheptanoyl)-4-piperidinyl]-1H-indole hydrochloride was obtained according to a similar manner to that of Example 7.

Mass: m/z 328(M-HCl+H)⁺

### EXAMPLE 33

3-[1-(7-Aminoheptanoyl)-4-piperidinyl]-1H-indole hydrochloride (1.9 g) was dissolved in diluted aqueous solution of sodium hydrogencarbonate and the solution was extracted with a mixture of methylene chloride and methanol(10:1). The organic layer was washed with brine and dried over magnesium sulfate. The organic layer was evaporated in vacuo and azeotroped with toluene. Lithium aluminum hydride (0.16 g) and THF (20 ml) were combined under nitrogen atmosphere. The solution of the residue described above in THF (20 ml) was added to the mixture and the reaction mixture was stirred for 1 h. Water (0.16 ml), 15% solution of sodium hydroxide (0.16 ml) and water (0.48 ml) were added to the mixture in order and the mixture was filtered. The filtrate was washed with THF and evaporated in vacuo. The residue was dissolved in AcOEt and the solution was washed with brine. The organic layer was dried over magnesium sulfate and evaporated in vacuo to give 3-[1-(7-aminoheptyl)-4-piperidinyl]-1H-indole (0.82 g).

### EXAMPLE 34

3-[1-(7-tert-Butoxycarbonylaminoheptyl)-4-piperidinyl]-1H-indole was obtained according to a similar manner to that of Example 1.

Mass: m/z 414(M+H)⁺

### EXAMPLE 35

The following compounds were obtained according to a similar manner to that of Example 16.

| Example | Chemical structure | Mass(m/z) or ¹H-NMR(δ) |
|---|---|---|
| 35-(1) | 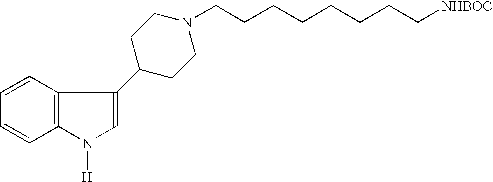 | 428(M + H)⁺ |
| 35-(2) | 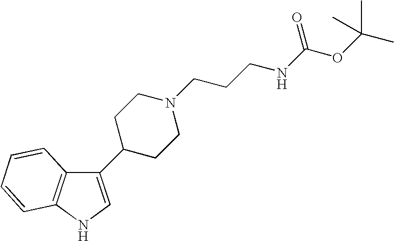 | (Solvent: DMSO-d₆) 7.53(1H, d, J=8Hz), 7.32(1H, d, J=8Hz), 7.02-7.08(2H, m), 6.94-6.97(1H, m), 6.83-6.86(1H, m), 2.92-2.99(4H, m), 2.70-2.76(1H, m), 2.37-2.43(2H, m), 1.90-2.05(4H, m), 1.55-1.73(4H, m), 1.378(9H, s) |

### EXAMPLE 36

3-[1-(5-Aminopentyl)-4-piperidinyl]-1H-indole, which was obtained according to a similar manner to that of Example 29, was dissolved in a mixture of EtOH and 4N-solution of hydrogen chloride in AcOEt. The mixture was evaporated in vacuo to give 3-[1-(5-Aminopentyl)-4-piperidinyl]-1H-indole-dihydrochloride.

### EXAMPLE 37

The following compounds were obtained according to a similar manner to that of Example 7.

| Example | Chemical structure | Mass(m/z) |
|---|---|---|
| 37-(1) | 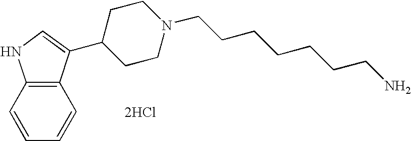 | 314(M − 2HCl + H)⁺ |
| 37-(2) | 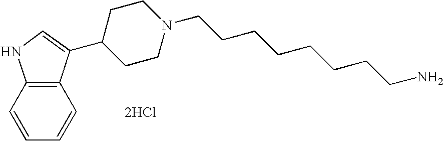 | 328(M − 2HCl + H)⁺ |
| 37-(3) | 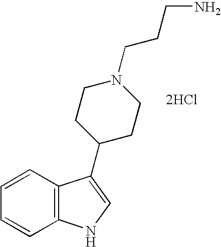 | 258(M − 2HCl + H)⁺ |

### EXAMPLE 38-(1)

Dioxane (25 ml) and acetyl chloride (1.60 ml) were combined under nitrogen atmosphere. Pyridine (3.62 ml) was added to the mixture at 20-25° C., then the mixture was stirred at 20-25° C. for 10 minutes. A solution of 5-(benzyloxy)-1H-indole (5.0 g) in dioxane (10 ml) was added thereto and the reaction mixture was heated at 35-45° C. for 30 minutes. Water (150 ml) was added to the mixture and the mixture was extracted with methylene chloride (150 ml). The combined organic layer was washed with 1N- hydrochloric acid, water and brine, and dried over magnesium sulfate. The solution was evaporated in vacuo and the residue was purified with silica gel chromatography (methylene chloride/hexane: 50/1 to methylene chloride/methanol: 10/1 elution) to give 3-(1-acetyl-1,4-dihydro-4-pyridyl)-5-(benzyloxy)-1H-indole (2.86 g).

Mass: m/z 345(M+H)⁺

### EXAMPLE 38-(2)

3-(1-Acetyl-1,4-dihydro-4-pyridyl)-5-(benzyloxy)-1H-indole (1.40 g), ethanol (70 ml) and platinum(IV) oxide (0.14 g) were combined under hydrogen atmosphere (latm). The reaction mixture was heated at 45-50° C. for 4.5 hours. Methylene chloride was added to the reaction mixture. The combined mixture was filtered and evaporated in vacuo. The residue was crystallized with ethanol and the crystal was collected by filtration and washed with ethanol to give 3-(1-acetyl-4-piperidinyl)-5-(benzyloxy)-1H-indole (1.13 g).

Mass: m/z 349(M+H)⁺

### EXAMPLE 38-(3)

3-(1-Acetyl-4-piperidinyl)-5-(benzyloxy)-1H-indole (1.12 g), 1N-aqueous solution of sodium hydroxide (16 ml) and ethanol (16 ml) were combined and the mixture was refluxed for 22 hours. Ethanol and water were added to the reaction mixture. The combined mixture was filtered and evaporated in vacuo. The residue was collected by filtration and washed with ethanol to give 5-(benzyloxy)-3-(4-piperidinyl)-1H-indole (0.98 g).

Mass: m/z 307(M+H)⁺

### EXAMPLE 38-(4)

5-(Benzyloxy)-3-(4-piperidinyl)-1H-indole (153 mg), ammonium formate (79 mg), ethanol (3 ml), water (0.3 ml) and 10% palladium on carbon (50% wet, 60 mg) were combined under hydrogen atmosphere (latm). The reaction mixture was refluxed for 1.5 hours then filtered. The filtrate was evaporated in vacuo. The residue was crystallized with ethanol and the crystal was collected by filtration and washed with ethanol to give 5-hydroxy-3-(4-piperidinyl)-1H-indole (60 mg).

Mass: m/z 217(M+H)⁺

### EXAMPLE 39

3-(1-Acetyl-4-piperidinyl)-5-hydroxy-1H-indol (0.34 g) was obtained according to a similar manner to that of Example 38-(4) by using 3-(1-acetyl-1,4-dihydro-4-pyridyl)-5-(benzyloxy)-1H-indole as a starting compound.

Mass: m/z 259(M+H)⁺

### EXAMPLE 40-(1)

5-(Benzyloxy)-1H-indole (25 g), 4,4-piperidinediol hydrochloride (25.8 g), potassium hydroxide (18.8 g) and methanol (250 ml) were combined under nitrogen atmosphere. The reaction mixture was refluxed for 19 h, and cooled to room temperature. Chloroform and water were added to the mixture, and the precipitate was collected by filtration to give 5-(benzyloxy)-3-(1,2,3,6-tetrahydro-4-pyridyl)-1H-indole (25.0 g).

Mass: m/z 305(M+H)⁺

### EXAMPLE 40-(2)

5-(Benzyloxy)-3-(1,2,3,6-tetrahydro-4-pyridyl)-1H-indole (22 g), tert-butyl-(7-bromoheptyl)carbamate (21.3 g), sodium iodide (10.8 g), Et₃N (20.1 ml) and DMF (220 ml) were combined under nitrogen atmosphere. The mixture was stirred at 60° C. for 20 h. After cooled to room temperature, the mixture was poured into water, and extracted with ethyl acetate. The combined organic layer was washed with brine, and dried over magnesium sulfate, and evaporated in vacuo. The residue was purified with NH-silica gel column chromatography (AcOEt: hexane=2:1 to AcOEt elution) to give 3-[1-(7-tert-butoxycarbonylaminoheptyl)-1,2,3,6-tetrahydro-4-pyridyl]-5-(benzyloxy)-1H-indole (36.4 g) as an brown oil.

Mass: m/z 518(M+H)⁺

### EXAMPLE 40-(3)

3-[1-(7-tert-Butoxycarbonylaminoheptyl)-1,2,3,6-tetrahydro-4-pyridyl]-5-(benzyloxy)-1H-indole (35.5 g), ammonium formate (13 g), 10% palladium on carbon (7.13 g), water (70 ml) and DMF (220 ml) were combined under nitrogen atmosphere. The reaction mixture was refluxed for 2 h. After cooled, the mixture was filtered and the filtrate was concentrated in vacuo. The residue was purified with NH-silica gel column chromatography (methanol chloroform=1:20 elution). The fractions contained the product were concentrated in vacuo. The residue was recrystallized from ethanol and water to give 3-[1-(7-tert-butoxycarbonylaminoheptyl)-4-piperidinyl]-5-hydroxy-1H-indole (13.4 g) as a white solid.

Mass: m/z 430(M+H)⁺

### EXAMPLE 41

3-[1-(7-tert-Butoxycarbonylaminoheptyl)-4-piperidinyl]-5-hydroxy-1H-indole (24 mg) was dissolved in methanol (0.5 ml). To the solution, a 2M solution of trimethylsilyldiazomethane in hexane (0.1 ml) was added under cooling with ice bath. Diisopropylethylamine (0.05 ml) was added thereto. The nixture was stirred at room temperature for 3 h, then acetic acid (0.1 ml) was added to the mixture. The solution was evaporated in vacuo. The residue was dissolved in ethyl acetate (5 ml) and the solution was washed with water and brine. The solution was dried over sodium sulfate and evaporated in vacuo. The residue was purified with NH silica gel chromatography (methanol/chloroform: 5/95 elution) to give 3-[1-(7-tert-butoxycarbonylaminoheptyl)-4-piperidinyl]-5-methoxy-1H-indole (19 mg) as a colourless oil.

Mass: m/z 444(M+H)⁺

### EXAMPLE 42-(1)

A mixture of 5-(benzyloxy)-3-(1,2,3,6-tetrahydro-4-pyridyl)-1H-indole (6 g), 7-phthalimidoheptyl bromide(6.39 g), triethylamine (5.49 ml) and sodium iodide (2.95 g) in DMF (60 ml) was stirred at room temperature for 12 h. The mixture was poured into water and extracted with ethyl acetate. The organic layer was washed with brine and dried over sodium sulfate. The solution was concentrated in vacuo to give 5-(benzyloxy)-3-[1-(7-phthalimidoheptyl)-1,2,3,6-tetrahydro-4-pyridyl]-1H-indole (log) as a yellow oil.

Mass: m/z 548(M+H)⁺

### EXAMPLE 42-(2)

A mixture of 5-(benzyloxy)-3-[1-(7-phthalimidoheptyl)-1,2,3,6-tetrahydro-4-pyridyl]-1H-indole (log) and ammonium formate (8.64 g) was dissolved in a mixture of EtOH (100 ml) and water (10 ml). To the mixture, 10% palladium on carbon (100 mg) was added. The mixture was stirred at 60° C. for 12 h. The reaction mixture was filtered and the filtrate was washed with a mixture of EtOH and chloroform. The filtrate was concentrated in vacuo. The residue was purified with silica gel column chromatography (MeOH/chloroform=1/10 to 1/1 elution) to give 5-hydroxy-3-[1-(7-phthalimidoheptyl)-4-piperidinyl]-1H-indole (4 g) as a pink powder.

Mass: m/z 460(M+H)⁺

### EXAMPLE 42-(3)

A mixture of 5-hydroxy-3-[1-(7-phthalimidoheptyl)-4-piperidinyl]-1H-indole (4 g) and hydrazine hydrate in EtOH (40 ml) was refluxed for 4 h. The mixture was filtered and the filtrate was evaporated in vacuo to give 3-[1-(7-aminoheptyl)-4-piperidinyl]-5-hydroxy-1H-indole (2.5 g).

### EXAMPLE 42-(4)

To a solution of 3-[1-(7-aminoheptyl)-4-piperidinyl]-5-hydroxy-1H-indole (2.5 g) in EtOH, a solution of 4N-hydrogen chloride in ethyl acetate (1.89 ml) was added at 0° C. The reaction mixture was stirred at room temperature for 30 min, then the mixture was concentrated in vacuo. The residue was washed with ether, and dried under reduced pressure at 45° C. for 9 h to give 3-[1-(7-aminoheptyl)-4-piperidinyl]-5-hydroxy-1H-indole dihydrochloride (1.7 g) as a yellow powder.

Mass: m/z 330(M-2HCl+H)⁺

### EXAMPLE 43

3-[1-(7-Aminoheptyl)-4-piperidinyl]-5-hydroxy-1H-indole dihydrochloride (30 mg) was dissolved in a mixture of pyridine (1 ml) and N,N-dimethylacetamide (0.5 ml). Acetic anhydride (0.07 ml) was added thereto. The mixture was stirred for 3 h, then AcOEt was added thereto. The mixture was washed with water, dried over sodium sulfate. The mixture was evaporated in vacuo. The residue was purified with NH silica gel chromatography (chloroform to chloroform/methanol: 98/2 elution) to give 3-[1-(acetyl-aminoheptyl)-4-piperidinyl]-5-hydroxy-1H-indole (7 mg) as a white powder.

Mass: m/z 372(M+H)⁺

### EXAMPLE 44

3-[1-(7-Bezoylaminoheptyl)-4-piperidinyl]-5-hydroxy-1H-indole was obtained according to a similar manner to that of Example 43.

Mass: m/z 434(M+H)⁺

### EXAMPLE 45-(1)

6-(Benzyloxy)-1H-indole (4.80 g), 4,4-piperidinediol hydrochloride (6.60 g), potassium hydroxide (4.26 g) and MeOH (50 ml) were combined and the mixture was refluxed for 2 days. After the mixture was cooled, water (250 ml) was added to the mixture and the mixture was stirred for 2 h. The solid in the mixture was collected by filtration and washed with water to give 6-(benzyloxy)-3-(1,2,3,6-tetrahydro-4-pyridyl)-1H-indole (6.80 g).

Mass: m/z 305(M+H)⁺

### EXAMPLE 45-(2)

6-(Benzyloxy)-3-(1-tert-butoxycarbonyl-1,2,3,6-tetrahydro-4-pyridyl)-1H-indole was obtained according to a similar manner to that of Example 1.

Mass: m/z 405(M+H)⁺

### EXAMPLE 45-(3)

6-(Benzyloxy)-3-(1-tert-butoxycarbonyl-1,2,3,6-tetrahydro-4-pyridyl)-1-(4-tert-butyl-benzyl)-1H-indole was obtained according to a similar manner to that of Example 2.

Mass: m/z 551(M+H)⁺

### EXAMPLE 45-(4)

6-(Benzyloxy)-3-(1-tert-butoxycarbonyl-1,2,3,6-tetrahydro-4-pyridyl)-1-(4-tert-butylbenzyl)-1H-indole (0.80 g), 10% palladium on carbon (0.16 g), EtOH (16 ml), and THF (16 ml) were combined and the mixture was stirred for 2 h under hydrogen atmosphere. The reaction mixture was filtered and the filtrate was evaporated in vacuo. The residue was purified with silica gel chromatography (hexane/AcOEt=6/1 to 4/1 elution) to give 6-hydroxy-3-[(1-tert-butoxycarbonyl)-4-piperidinyl]-1-(4-tert-butylbenzyl)-1H-indole (0.26 g) and 6-benzyloxy-3-[(1-tert-butoxycarbonyl)-4-piperidinyl]-1-(4-tert-butylbenzyl)-1H-indole (0.27 g).

6-Hydroxy-3-[( 1-tert-butoxycarbonyl)-4-piperidinyl]-1-(4-tert-butylbenzyl)-1H-indole:

Mass: m/z 463(M+H)⁺

6-Benzyloxy-3-[(1-tert-butoxycarbonyl)-4-piperidinyl]-1-(4-tert-butylbenzyl)-1H-indole:

Mass: m/z 553(M+H)⁺

### EXAMPLE 46

The following compounds were obtained according to a similar manner to that of Example 45.

| Ex- | | |
|---|---|---|
| am- | | Mass(m/z) or |
| ple | Chemical structure | ¹H-NMR(δ) |
| 46- (1) | 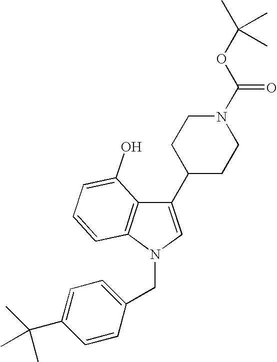 | 363 (M − Boc + H)⁺ |
| 46- (2) | 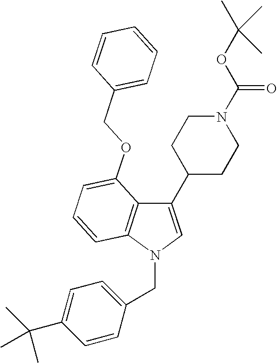 | 453 (M − Boc + H)⁺ |
| 46- (3) | 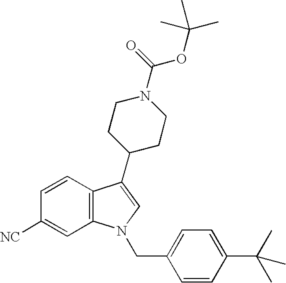 | N.D |
| 46- (4) | 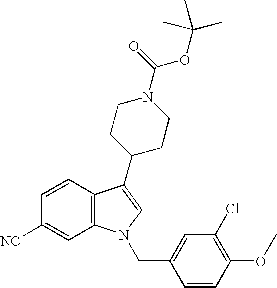 | (Solvent: DMSO-d₆) 7.68(1H, d, J=8Hz), 7.56(1H, s), 7.33(1H, dd, J=2Hz, 8Hz), 7.14(1H, d, J=2Hz), 7.05(1H, s), 6.96(1H, dd, J=2Hz, 8Hz), 6.87(1H, d, J=8Hz), 5.20(2H, s), 4.17-4.33(2H, m), 3.89(3H, s), |
| #2.82-3.07(3H, m), 1.96-2.07(2H, m), 1.59-1.75(2H, m), 1.48(9H, s) | | |
| 46- (5) | 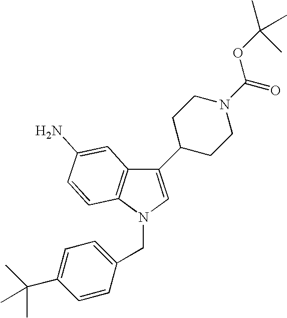 | 462(M + H)⁺ |
| 46- (6) | 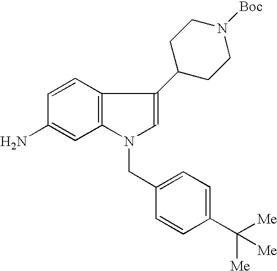 | 462(M + H)⁺ |

### EXAMPLE 47

6-Amino-3-[(1-tert-butoxycarbonyl)-4-piperidinyl]-1-(4-tert-butylbenzyl)-1H-indole (500 mg) was dissolved in methylene chloride (5 ml). To the solution, Et3N (166 μl) and benzoyl chloride (138 μl) were added and the mixture was stirred for 10 min. To the reaction mixture, methylene chloride was added and the mixture was washed with water and brine. The organic layer was dried over magnesium sulfate and evaporated in cacuo. The residue was purified with silica gel chromatography (methylene chloride/methanol=200/1 to 50/1 elution) to give 6-(N-benzoylamino)-3-[(1-tert-butoxycarbonyl)-4-piperidinyl]-1-(4-tert-butylbenzyl)-1H-indole (610 mg)

Mass: m/z 466(M-Boc+H)⁺

### EXAMPLE 48

6-(N-Benzoylamino) -3-1(1-tert-butoxycarbonyl) -4-piperadinyl]-1-(4-tert-butylbenzyl)-1H-indole (300 mg) was dissolved in DMF (3 ml) and sodium hydride (60% 42 mg) was added to the solution. After the mixture was stirred for 10 min, iodomethane (165 μl) was added thereto and then the mixture was stirred for 30 min. To the mixture, toluene was added and the mixture was washed with water and brine. The organic layer was dried over magnesium sulfate and evaporated in vacuo. The residue was purified with silica gel chromatography (methylene chloride/methanol:200/1 to 50/1 elution) to give 6-(N-benzyol-N-methylamino)-3-[(1-tert-butoxycarbonyl)-4-piperidinyl]-1-(4-tert-butylbenzyl)-1H-indole (300 mg).

Mass: m/z 580(M+H)⁺

### EXAMPLE 49

The following compounds were obtained according to a similar manner to that of Examples 45 and 7.

| Example | Chemical structure | Mass(m/z) |
|---|---|---|
| 49-(1) | 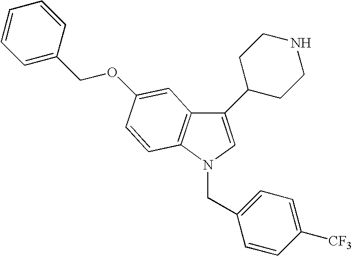 | 466(M + H)⁺ |
| 49-(2) | 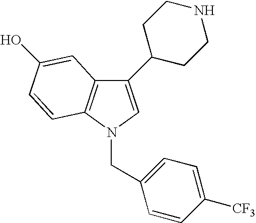 | 375(M + H)⁺ |
| 49-(3) | 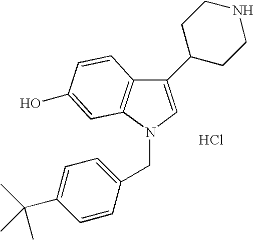 | 363(M − HCl + H)⁺ |
| 49-(4) | 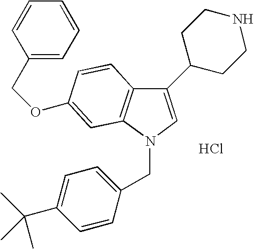 | 453(M − HCl + H)⁺ |
| 49-(5) | 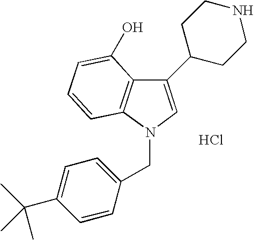 | 363(M − HCl + H)⁺ |
| 49-(6) | 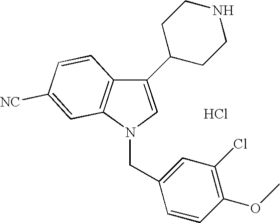 | 380(M − HCl + H)⁺ |
| 49-(7) | 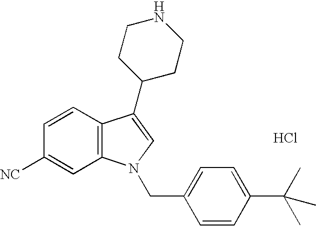 | 372(M − HCl + H)⁺ |
| 49-(8) | 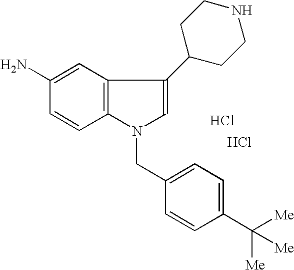 | 362(M − 2HCl + H)⁺ |

### EXAMPLE 50

6-Hydroxy-3-[(1-tert-butoxycarbonyl)-4-piperidinyl]-1-(4-tert-butylbenzyl)-1H-indole (46 mg), sodium hydride (60% 8 mg) and DMF (0.5 ml) was combined and stirred for 10 min. To the mixture, 4-(tert-butyl)benzyl bromide (55 μl) was added and the mixture was stirred for 1.5 h. Toluene and water were added thereto. After separated form the organic layer, the aqueous layer was extracted with toluene. The organic layers were combined and washed with brine. The organic layer was evaporated in vacuo and the residue was purified with silica gel chromatography (hexane/AcOEt=9/1 to 6/1 elution) to give 6-(4-tert-butylbenzyloxy)-3-[1-(tert-butoxycarbonyl)-4-piperidinyl]-1-(4-tert-butylbenzyl)-1H-indole (59 mg). To the solution of 6-(4-tert-butylbenzyloxy)-3-[1-(tert-butoxycarbonyl)-4-piperidinyl]-1-(4-tert-butylbenzyl)-1H-indole in methylene chloride (0.6 ml), 4N hydrogen chloride in AcOEt (0.6 ml) was added and the mixture was stirred for 1.5 h. The mixture was evaporated in vacuo and the residue was collected by filteration to give 6-(4-tert-butylbenzyloxy)-3-(4-piperidinyl)-1-(4-tert-butylbenzyl)-1H-indole hydrochloride (48 mg).

Mass: m/z 509(M-HCl+H)⁺

### EXAMPLE 51

The following compounds were obtained according to a similar manner to that of Example 50.

| Example | Chemical structure | Mass(m/z) |
|---|---|---|
| 51-(1) | 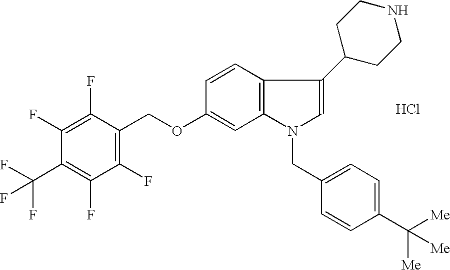 | 593(M − HCl + H)⁺ |
| 51-(2) | 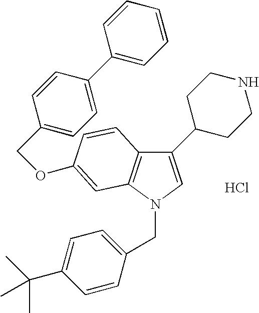 | 529(M − HCl + H)⁺ |
| 51-(3) | 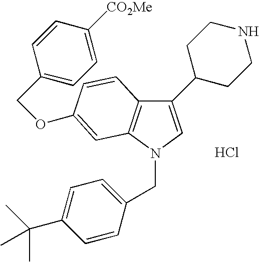 | 511(M − HCl + H)⁺ |
| 51-(4) | 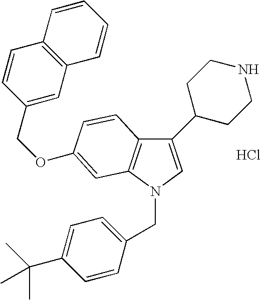 | 503(M − HCl + H)⁺ |
| 51-(5) | 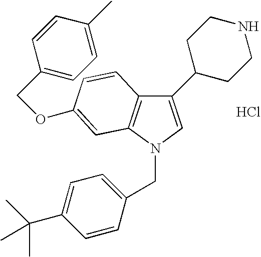 | 467(M − HCl + H)⁺ |
| 51-(6) | 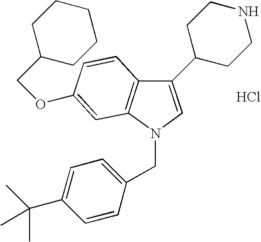 | 459(M − HCl + H)⁺ |
| 51-(7) | 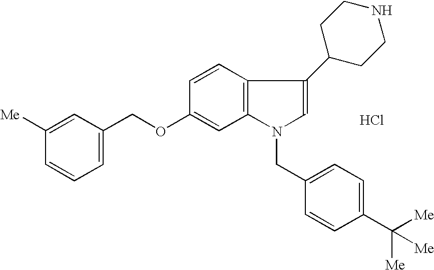 | 467(M − HCl + H)⁺ |
| 51-(8) | 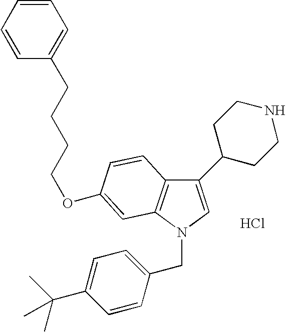 | 495(M − HCl + H)⁺ |
| 51-(9) | 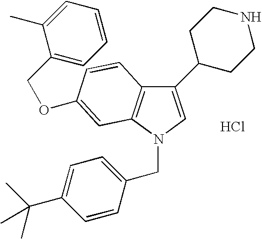 | 467(M − HCl + H)⁺ |
| 51-(10) | 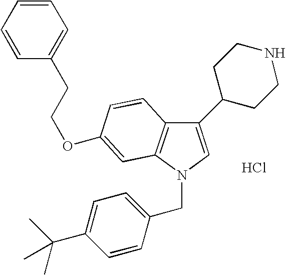 | 467(M − HCl + H)⁺ |
| 51-(11) | 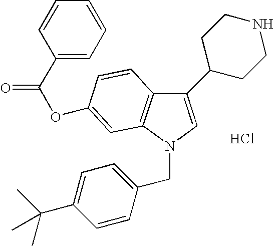 | 467(M − HCl + H)⁺ |

The following compounds were obtained according to a similar manner to that of Example 7.

| Ex- | | |
|---|---|---|
| ample | Chemical structure | Mass(m/z) |
| 51-(12) | 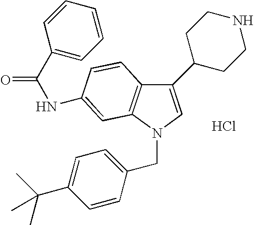 | 466(M − HCl + H)⁺ |
| 51-(13) | 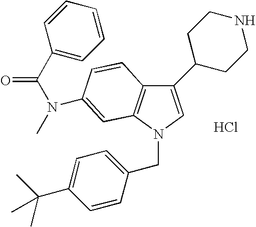 | 480(M − HCl + H)⁺ |

### EXAMPLE 52

1-(3-Chloro-4-methoxybenzyl)-6-cyano-3-(4-piperidinyl)-1H-indole hydrochloride (60 mg) was suspended in chloroform and the mixture was washed with saturated solution of sodium hydrogencarbonate. The organic layer was evaporated in vacuo and the residue was dissolved in mixed solution of methylene chloride (1 ml) and methanol (2 ml). To the solution, 37% formaldehyde solution (53.1 mg), sodium cyanoborohydride (24.7 mg) and acetic acid (5 drops) were added and the reaction mixture was stirred for 2 h. The mixture was diluted with chloroform and washed with water, saturated solution of sodium hydrogencarbonate and brine. The organic layer was dried over magnesium sulfate and evaporated in vacuo. The residue was purified with preparative TLC (silica gel,chloroform/methanol/conc ammonia solution=10/1/0.1) to give 1-(3-chloro-4-methoxybenzyl)-6-cyano-3-( 1-methyl-4-piperidinyl)-1H-indole (42.4 mg).

Mass: m/z 395(M+H)⁺

### EXAMPLE 53

1-(3-Chloro-4-methoxybenzyl)-6-cyano-3-(4-piperidinyl)-1H-indole hydrochloride (50 mg), triethylamine (48.6 mg), acetic anhydride (24.5 mg) and dried methylene chloride (1 ml) were combined and stirred for 4 h. Water was added to the mixture and the organic layer was separated. The organic layer was washed with 1N-hydrochloric acid, water, saturated solution of sodium hydrogencarbonate and brine and dried over magnesium sulfate. The organic layer was evaporated in vacuo to give 3-(1-acetyl-4-piperidinyl)-6-cyano-1-(3-chloro-4-methoxybenzyl)-1H-indole (46.0 mg).

Mass: m/z 422(M+H)⁺

### EXAMPLE 54

1-(3-Chloro-4-methoxybenzyl)-6-cyano-3-(4-piperidinyl)-1H-indole hydrochloride (50 mg) was combined with dried methylene chloride (1 ml) under nitrogen atmosphere. To the mixture, dry triethylamine (48.6 mg) and methanesulfonyl chloride (20.6 mg) were added and the mixture was stirred for 15 h. Water and chloroform were added to the mixture and the organic layer was separated. The organic layer was washed with 1N-hydrochloric acid, water, saturated solution of sodium hydrogencarbonate, and brine and dried over magnesium sulfate and then evaporated in vacuo. The residue was crystallized with IPE and the crystal was collected by filtration to give 6-cyano-3-( 1-methylsulfonyl-4-piperidinyl)-1-(3-chloro-4-methoxybenzyl)-1H-indole (50.5 mg).

¹H-NMR (Solvent: DMSO-d₆)(δ) 8.17 (1H, s), 7.78 (1H, d, J=8 Hz), 7.71 (1H, s), 7.44 (1H, d, J=2 Hz) , 7.34 (1H, d, J=8 Hz), 7.25 (1H, dd, J=2 Hz, 8 Hz), 7.09 (1H, d, J=8 Hz), 5.36 (2 H, s), 3.80 (3H, s),3.73-3.84 (2H, m), 3.50-3.60 (1H, m), 2.84-3.05 (5H, m), 2.00-2.13 (2H, m), 1.62-1.78 (2H, m)

1. A method for preventing and/or treating a bone disease which comprises administering a compound of the formula [I]: 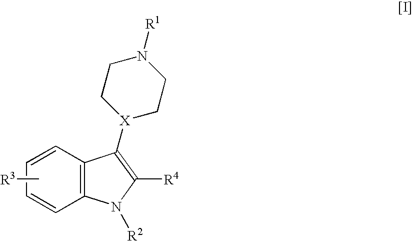
wherein
R¹ is hydrogen, acyl, lower alkyl, optionally substituted ar(lower)alkyl or a group of the formula:
-A-B,
in which A is alkylene having one to ten carbon atoms, and B is amino optionally substituted with acyl or lower alkyl,
R² is hydrogen, lower alkyl, cyclo(lower)alkyl(lower)alkyl, acyl, optionally substituted ar(lower)alkyl or optionally substituted heterocyclic-(lower)alkyl,
R³ is hydrogen, optionally substituted hydroxy, optionally substituted amino or cyano,
R⁴ is hydrogen or lower alkyl, and
X is CH or N,
or its pharmaceutically acceptable salt to human being or animals.

2. A method of claim 1, wherein
R¹ is
(1) hydrogen,
(2) acyl,
(3) lower alkyl,
(4) ar(lower)alkyl optionally substituted with lower alkoxy, or
(5) a group of the formula:
-A-B
in which,
A is alkylene having one to ten carbon atoms, and
B is amino optionally substituted with acyl or lower alkyl,
R² is
(1) hydrogen,
(2) lower alkyl,
(3) cyclo(lower)alkyl(lower)alkyl,
(4) acyl,
(5) ar(lower)alkyl optionally substituted with one or more substituents selected from the group consisting of
lower alkyl,
halo(lower)alkyl,
nitro,
amino,
halogen,
cyano,
hydroxy,
lower alkoxy,
lower alkylthio,
aryl optionally substituted with lower alkyl or
halo(lower)alkyl,
acyl,
ar(lower)alkyl,
lower alkylenedioxy,
aryloxy,
ar(lower)alkenyl, and
ar(lower)alkoxy, or
(6) heterocyclic(lower)alkyl optionally substituted with one or more substituents selected from the group consisting of
lower alkyl,
halo(lower)alkyl,
nitro,
amino,
halogen,
cyano,
hydroxy,
lower alkoxy,
lower alkylthio,
aryl optionally substituted with lower alkyl or
halo(lower)alkyl,
acyl,
ar(lower)alkyl,
lower alkylenedioxy,
aryloxy,
ar(lower)alkenyl, and
ar(lower)alkoxy,
R³ is
(1) hydrogen,
(2) hydroxy,
(3) aroyloxy or ar(lower)alkoxy, each of which is optionally substituted with one or more substituents selected from the group consisting of
halogen,
acyl,
aryl,
lower alkyl, and
halo(lower)alkyl,
(4) lower alkoxy,
(5) cyclo(lower)alkyl(lower)alkoxy,
(6) amino optionally substituted with lower alkyl or acyl, or
(7) cyano.

3. A method of claim 2, wherein
R¹ is
(1) hydrogen,
(2) lower alkanoyl,
(3) lower alkoxycarbonyl,
(4) amino(lower)alkanoyl,
(5) lower alkoxycarbonylamino(lower)alkanoyl,
(6) lower alkylsulfonyl,
(7) lower alkyl,
(8) phenyl(lower)alkyl optionally substituted with lower alkoxy, or
(9) a group of the formula:
-A-B
wherein A is alkylene having one to ten carbon atoms, and B is amino optionally substituted with one or two substituents selected from the group consisting of
lower alkyl,
lower alkanoyl,
lower alkoxycarbonyl,
benzoyl, and
phthaloyl,
R² is
(1) hydrogen,
(2) lower alkyl,
(3) cyclo(lower)alkyl(lower)alkyl,
(4) benzoyl optionally substituted with lower alkyl or halo(lower)alkyl,
(5) lower alkylsulfonyl,
(6) phenylsulfonyl,
(7) phenyl(lower)alkyl, naphthyl(lower)alkyl or anthryl-(lower)alkyl, each of which is optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl optionally substituted with lower alkyl or
halo(lower)alkyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
(8) quinolyl(lower)alkyl or oxadiazolyl(lower)alkyl, each of which is optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl optionally substituted with lower alkyl or
halo(lower)alkyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
R³ is
(1) hydrogen,
(2) hydroxy,
(3) benzoyloxy,
(4) phenyl(lower)alkoxy or naththyl(lower)alkoxy, each of which is optionally substituted with one or more substituents selected from the group consisting of
halogen,
phenyl,
lower alkyl,
halo(lower)alkyl, and
lower alkoxycarbonyl,
(5) lower alkoxy,
(6) cyclo(lower)alkyl(lower)alkoxy,
(7) amino optionally substituted with lower alkyl or benzoyl, or
(8) cyano.

4. A method of claim 3, wherein
R¹ is hydrogen or lower alkoxycarbonyl,
R² is phenyl(lower)alkyl optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
R³ is
(1) hydrogen,
(2) hydroxy,
(3) benzoyloxy,
(4) phenyl(lower)alkoxy optionally substituted with one or more substituents selected from the group consisting of
halogen,
phenyl,
lower alkyl,
halo(lower)alkyl, and
lower alkoxycarbonyl,
(5) lower alkoxy,
(6) cyclo(lower)alkyl(lower)alkoxy,
(7) amino optionally substituted with lower alkyl or benzoyl, or
(8) cyano,
R⁴ is hydrogen, and
X is CH.

5. A method of claim 3, wherein
R¹ is a group of the formula:
-A-B
in which,
A is alkylene having one to ten carbon atoms, and
B is amino optionally substituted with one or two substituents selected from the group consisting of
lower alkyl,
lower alkanoyl,
lower alkoxycarbonyl,
benzoyl, and
phthaloyl,
R² is
(1) hydrogen,
(2) lower alkyl,
(3) cyclo(lower)alkyl(lower)alkyl,
(4) benzoyl optionally substituted with lower alkyl or halo(lower)alkyl,
(5) lower alkylsulfonyl,
(6) phenylsulfonyl,
(7) phenyl(lower)alkyl optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
R³ is
(1) hydrogen,
(2) hydroxy,
(3) benzoyloxy,
(4) phenyl(lower)alkoxy optionally substituted with one or more substituents selected from the group consisting of
halogen,
phenyl,
lower alkyl,
halo(lower)alkyl, and
lower alkoxycarbonyl,
(5) lower alkoxy,
(6) cyclo(lower)alkyl(lower)alkoxy,
(7) amino optionally substituted with lower alkyl or benzoyl, or
(8) cyano,
R⁴ is hydrogen, and
X is CH.

6. A method of claim 1, wherein the bone disease is selected from the group consisting of low bone mass, osteoporosis, bone fracture, bone refracture, bone defect, osteomalacia, Behcet's syndrome in bone, osteotomy, cartilage defect, Paget's disease, rigid myelitis, chronic rheumatoid arthritis, chronic rheumatoid arthritis involving a cartilage, osteoarthritis, osteoarthritis of the knee, osteoarthritis involving cartilage, osteoarthritis of a knee involving cartilage, bone loss associated with periodontitis, prosthetic ingrowth, alveolar or mandibular bone loss, childhood idiopathic bone loss and secondary osteoporosis.

7. A compound of the formula [If]: 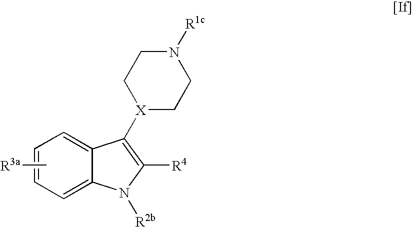
wherein
R^{1c} is hydrogen or acyl,
R^{2b} is optionally substituted ar(lower)alkyl,
R^{3a} is hydrogen, hydroxy, lower alkoxy, cyano, amino or acylamino,
R⁴ is hydrogen or lower alkyl, and
X is CH or N,
or its pharmaceutically acceptable salt.

8. A compound according to claim 7, wherein
R^{1c} is hydrogen or lower alkoxycarbonyl,
R^{2b} is phenyl(lower)alkyl optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
R^{3a} is
(1) hydrogen,
(2) hydroxy,
(3) lower alkoxy,
(4) amino optionally substituted with benzoyl, or
(5) cyano,
R⁴ is hydrogen, and
X is CH.

9. A compound according to claim 8, wherein
R^{2b} is phenyl(lower)alkyl optionally substituted with one or more substituents selected from the group consisting of
lower alkyl,
lower alkoxy,
lower alkylthio,
lower alkylsulfonyl,
halogen,
lower alkoxycarbonyl,
nitro,
halo(lower)alkyl, and
lower alkylenedioxy,
R^{3a} is hydrogen or cyano.

10. A pharmaceutical composition comprising a compound of claim 7, as an active ingredient, in association with a pharmaceutically acceptable, substantially non-toxic carrier or exipient.

11. A method for preventing and/or treating a bone disease which comprises administering the compound of claim 7 to human being or animals.

12. A compound of the formula [Ig]: 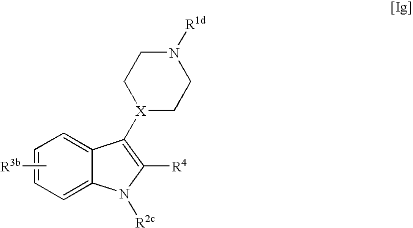
wherein
R^{1d} is a group of the formula
-A¹-B¹
in which
A¹ is alkylene having six to ten carbon atoms, and
B¹ is amino optionally substituted with acyl or lower alkyl,
R^{2c} is hydrogen or optionally substituted ar(lower)alkyl,
R^{3b} is hydrogen, hydroxy or lower alkoxy,
R⁴ is hydrogen or lower alkyl, and
X is CH or N,
or its pharmaceutically acceptable salt.

13. A compound according to claim 12, wherein
R^{1d} is a group of the formula:
-A¹-B¹
in which
A¹ is alkylene having seven to ten carbon atoms, and
B¹ is amino optionally substituted with one or two substituents selected from the group consisting of
lower alkyl,
lower alkanoyl,
lower alkoxycarbonyl,
benzoyl, and
phthaloyl,
R^{2c} is
(1) hydrogen,
(2) phenyl(lower)alkyl optionally substituted with one or more substituents selected from the group consisting of
lower alkyl,
halo(lower)alkyl,
nitro,
amino,
halogen,
hydroxy,
cyano,
lower alkoxy,
lower alkylthio,
phenyl,
lower alkoxycarbonyl,
lower alkylsulfonyl,
carboxy,
N-phenylcarbamoyl,
N-phenyl-N-(lower)alkylcarbamoyl,
phenyl(lower)alkyl,
lower alkylenedioxy,
phenoxy,
phenyl(lower)alkenyl, and
phenyl(lower)alkoxy,
R⁴ is hydrogen, and
X is CH.

14. A compound according to claim 13, wherein
R^{1d} is a group of the formula:
-A¹-B¹
in which
A¹ is straight alkylene having seven to ten carbon atoms, and
B¹ is lower alkanoylamino or lower alkoxycarbonylamino,
R^{2c} is hydrogen.

15. A pharmaceutical composition comprising a compound of claim 12, as an active ingredient, in association with a pharmaceutically acceptable, substantially non-toxic carrier or exipient.

16. A method for preventing and/or treating a bone disease which comprises administering the compound of claim 12 to human being or animals.
